(19) United States
(12) Patent Application Publication
(10) Pub. No.: US 2024/0400589 A1
(43) Pub. Date: Dec. 5, 2024

(54) 4-AMINOPHENYLPHOSPHORYLCHOLINE COMPOUNDS FOR BLOCKING C-REACTIVE PROTEIN

(71) Applicant: Pentracor GmbH, Hennigsdorf (DE)

(72) Inventors: Rudolf Kunze, Flieth-Stegeliz (DE); Birgit Vogt, Berlin (DE); Ahmed Sheriff, Berlin (DE); Stephan Mattecka, Berlin (DE)

(21) Appl. No.: 18/702,341

(22) PCT Filed: Oct. 19, 2023
(86) PCT No.: PCT/EP2022/079069

§ 371 Date: Apr. 18, 2024

(30) Foreign Application Priority Data
Oct. 19, 2021 (EP) 21 203 495.3

(51) Int. Cl. C07F 9/09; A61K 31/688; A61K 45/06; C07F 9/6561

(52) U.S. Cl. CPC C07F 9/09; A61K 31/688; A61K 45/06; C07F 9/6561

(57) ABSTRACT

The present invention relates to compounds of the general formula (I) which bind to and/or neutralise C-reactive protein. The compounds according to the invention are particularly useful for the treatment and/or prevention of acute or chronic diseases associated with and/or caused by elevated CRP levels.

The present invention relates to compounds of the general formula (I) which bind to and/or neutralise C-reactive protein. The compounds according to the invention are particularly useful for the treatment and/or prevention of acute or chronic diseases associated with and/or caused by elevated CRP levels.

In human medicine, C-reactive protein (CRP) has been known for many years as the acute phase protein. Elevated CRP blood levels are observed in particular in inflammations of various causes. CRP is therefore one of the most important indicators of inflammatory disease processes. The formation of CRP takes place in the liver. Its synthesis is triggered hours after an injury and/or an infection and is limited in time. CRP formation depends on stimulation by pro-inflammatory cytokines, in particular interleukin-6.

CRP is a phylogenetically old molecule and widely distributed in the animal kingdom. It was first discovered in 1930 in the blood of people with acute bacterial infections. The physiologically dominant form is a pentamer consisting of identical subunits that are not covalently linked. With a molecular weight of approx. 125,000 Da, it is one of the larger plasma molecules. Concentrations of 0.2-3 mg/L blood are regarded as the normal range.

In the local microenvironment, in the presence of low pH and oxidative stress species (ROX), CRP undergoes a conformational change that enables complement binding (C1q), as well as the dissociation of the pentameric form (pCRP) into its monomers (mCRP). Both enable or intensify the local inflammatory processes. The dissociation of the pentamer is a localised process, which is thought to be promoted by lyso-phosphocholine. The monomeric CRP is said to play a special role in the destruction of the blood-retinal barrier (Mollins et al. Front. Immunol. 2018, 9:808). According to the authors, moderately elevated pCRP levels are in equilibrium with the locally pathologically more active mCRP. In an animal model (rat, ischaemia model), Braig et al. (Nat Commun; 2017, 8:14188) and Thiele et al. (Front Immunol; 2018, 9: art.6) showed that leukocyte rolling and adherence in capillary vessels of muscle tissue is increased by mCRP, but not by pCRP. The local molecular inflammatory cascade is probably intensified with the formation of mCRP. The authors assume that the conformational change of CRP is the key to understanding vascular inflammatory pathomechanisms in diseases triggered by vasoconstriction or occlusion (e.g. myocardial infarction, stroke).

CRP is one of the few plasma molecules whose concentration in the blood can increase more than a hundredfold. This is triggered by vascular occlusions such as heart attacks or strokes, but also by acute infection, septicaemia, burns, severe trauma, acute pancreatitis or surgery. The CRP concentration rises after one of the above-mentioned events with a delay of several hours. A peak is observed around 48 hours after a heart attack, for example, while the CRP level falls continuously in the days following. The individual CRP response rates, extent and duration vary greatly.

Independently of each other, the research groups led by Lucchesi (Barrett et al, J Pharmacol Exp Ther 2002; 303(3):1007-1013) and Pepys (Griselli et al, J Exp Med 1999; 190(12):1733-1740; Gill et al, J Cereb Blood Flow Metab 2004; 24(11):1214-1218) demonstrated a pro-oedematous and pronecrotic effect of CRP in myocardial infarction and stroke in rabbits and rats respectively. From an evolutionary perspective, in addition to a certain antibacterial effect, the function of CRP is the labelling of necrotic and pronecrotic cells in order to dispose of them with the help of complement proteins and phagocytes and to initiate tissue regeneration. This mechanism is indispensable for the healing of external wounds, but excessive CRP concentrations may be counterproductive for the healing of internal, aseptic wounds such as heart attacks or strokes, in other words: evolution is not adapted to this type of injury or wound.

The molecular mechanisms of the pro-inflammatory effect of CRP on the cell surface are roughly known. In the event of a local injury, acute inflammation follows. Three cell populations can temporarily develop at the cellular level: vital, reversibly defective and irreversibly defective cells. The transitions between the latter two are fluid. CRP is a trigger of cell death or necrosis, and the proportion of reversibly damaged cells that reach the “point of no return” probably depends on the amount of CRP on site.

The natural ligand of CRP, lysophosphatidylcholine (LPC), is practically not present in vital cells as a component of the cell membrane. However, if cell damage occurs, LPC is produced by a special phospholipase, another acute phase protein, on the cell surface by an enzyme. This is the secretory phospholipase A2 type IIa (sPLA2 IIa). The more CRP is present, the more CRP molecules bind to the newly formed LPC molecules. Cells in an infarct area are usually poorly supplied with oxygen and nutrients and therefore switch their metabolism from the respiratory chain to glycolysis, which leads to a depletion of energy equivalents. Without this energy restriction, the newly formed LPC would be repaired immediately.

Upon binding to the pronecrotic cell or LPC, CRP undergoes a conformational change and the inflammatory cascade is set in motion, which now enables the complement protein C1q to bind to CRP. The complement cascade then proceeds to C4 and factor H. The result is the invasion of monocytes and the induction of pro-inflammatory cytokines, which in turn stimulate the synthesis of CRP in the liver. The “clearance” of dead or no longer fully functional cells, e.g. by phagocytes, is an essential mechanism for the initiation of tissue regeneration and wound healing.

The normal value for CRP in the blood of humans varies from person to person, is on average around 0.8 mg CRP per litre of blood, but can rise to well over 100 mg CRP per litre of blood in the case of acute or chronic inflammatory reactions (e.g. bacterial infections, atherosclerosis, after a heart attack). As the half-life of CRP in the blood (approx. 19 hours) is constant and largely independent of the patient's state of health, the rate of CRP synthesis alone is responsible for regulating the CRP level in the blood (Pepys & Hirschfield, J. Clin. Invest., 2003, 111: 1805-1812). The greatly increased synthesis of CRP in acute pathological conditions therefore places special demands on therapeutic approaches to CRP removal from patients (high-risk patients or acute patients), as a considerable amount of CRP must be removed in order to reduce the CRP level in the blood to normal values. The damage to the heart after a heart attack or to the brain after a stroke is exacerbated by CRP and the subsequent complement effect.

Three main ways of neutralising the effect of CRP are known:
Inhibition of CRP synthesis
removing of CRP from the bloodstream
pharmacological blocking of CRP

Effective inhibition of CRP synthesis is possible with the help of antisense oligonucleotides, which inhibit CRP synthesis in the liver (Noveck et al., J Am Heart Assoc. 2014; doi.org/10.1161/JAHA.114001084). This was demonstrated in healthy volunteers who showed high plasma levels of CRP after an injection of endotoxin. However, the active substance ISIS-CRPRx was administered in 6 doses over 22 days in advance. Obviously, the oligonucleotide is not suitable for lowering acute CRP concentrations, which frequently occur in the case of heart attacks or strokes. CRP can be removed from the bloodstream or blood plasma by means of selective apheresis using a CRP adsorber. The practicability and efficiency of a CRP apheresis system in acute inflammation was demonstrated in an infarct model in pigs (Sheriff et al., Journal of Clinical Apheresis 2015; 30: 15-21. doi.org/10.1002/jca.21344). Using a CRP adsorber, the amount of CRP in the animals' blood was lowered after the infarction was triggered. The subsequent cardiological examination by MRI and histology showed a significantly smaller infarct area and less scar tissue in the treatment group compared to the control animals.

Previous findings from a pilot study in patients with myocardial infarction confirmed the positive effect of CRP apheresis (Ries et al. C-reactive protein apheresis as anti-inflammatory therapy in acute myocardial infarction: Results of the CAMI-1 study. Front Cardiovasc Med 2021; 8:591741). The infarct area is significantly smaller in patients with CRP apheresis, while the left ventricular ejection fraction (LVEF) increases significantly.

Lowering the CRP concentration in the blood is therefore likely to reduce the amount of CRP on ischaemic or pronecrotic cells (or in their immediate vicinity), e.g. in an infarct area. As a result, fewer binding sites are available for complement proteins. The only pre-damaged but reversible part of the affected inflammatory tissue is able to regenerate. Whether this regenerative process, caused by the reduction in CRP concentration, also takes place in tissues other than muscle cells is unknown, but probable.

In principle, the specific CRP adsorber can be used as a medical device wherever the removal of CRP is of therapeutic and clinical benefit in the course of a preferably acute inflammation or disease with high CRP levels. However, due to the medical technology requirements, the procedure cannot always be used where the reduction of functionally active CRP or CRP blood levels is desired. The use of extracorporeal apheresis procedures such as CRP apheresis, immunoadsorption or lipid apheresis requires a considerable amount of medical technology. This imposes a certain technical limitation on the practicability of the procedure, including CRP apheresis.

The pharmacological blocking of CRP is the third therapeutic option for curbing the effect of CRP on pre-damaged cells or tissue, as is known from heart attacks or strokes. The active ingredient 1,6-bis(phosphocholine)-hexane blocks CRP in vivo. Using the active substance derived from the natural ligand phosphocholine, an experiment in rats showed that blocking CRP leads to a reduced volume of artificially induced myocardial infarction (Pepys et al., Nature 2006; 440(7088):1217-1221). However, the CRP blocker was administered before the myocardial infarction was triggered and not immediately afterwards. For the acute treatment of a heart attack, the active substance should take effect immediately or at least within a few minutes after the heart attack. An active substance/medication that directly targets CRP is currently not available.

The Japanese patent JP 63-190888 A discloses echinosporin derivatives (XK 213) having a cytostatic effect. The U.S. Pat. No. 5,661,138 describes the preparation and use of alkyl- and alkenyl-substituted phenylphosphoethanolamines. These phosphocholine derivatives also showed an anti-inflammatory effect.

The objective of the present invention is therefore to provide a medicament for binding and/or neutralising CRP in vivo, in particular in the bloodstream, in particular for the treatment and/or prevention of acute or chronic diseases associated with and/or caused by an elevated CRP level.

This objective is solved by the technical teaching of the independent claims of the present invention. Further advantageous embodiments of the invention are shown in the dependent claims, the description and the examples.

Surprisingly, the inventors have found that a compound of the general formula (I) binds CRP and is thus suitable for binding and/or neutralising in particular free, dissolved CRP in the blood. Thus, said compound is particularly suitable for the treatment and/or prevention of acute or chronic diseases associated with and/or caused by an elevated CRP level.

### BRIEF DESCRIPTION

The present invention relates to a compound of general formula (I):

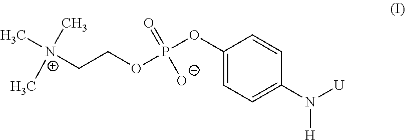

wherein U is selected from —P₁—R₁, —CO—Z₁, —P₂—Z₂ or

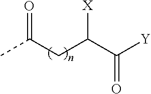

wherein n is an integer selected from 1, 2 and 3;
X represents —NH₂, —NH—P₁—R₁, —NH—CO—Z₁ or —NH—P₁—Z₁;
Y represents —OH, —NH₂, —P₂—R₂, —NH—Z₂ or —P₂—Z₂;
R₁ is selected from —H, —CH₃, —CH₂CH₃, —CO—CH₃ and —CO—CH₂CH₃;
R₂ is selected from —OH, —OCH₃, —OCH₂CH₃, —CH₃, and —NH₂;
P₁ is selected from -Q₁-, -Q₁-Q₂-, -Q₁-Q₂-Q₃- and -Q₁-Q₂-Q₃-Q₄-;
P₂ is selected from -Q₅-, -Q₅-Q₆-, -Q₅-Q₆-Q₇- and -Q₅-Q₆-Q₇-Q₈-;

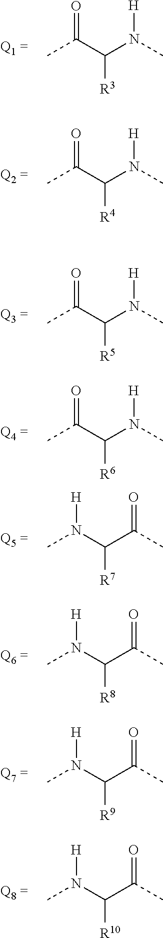

wherein R³, R⁴, R⁵, R⁶, R⁷, R⁸, R⁹ and R¹⁰ represent independently of each other —H, —CH₃, —CH₂OH, —CH₂SH, —COOH, —CONH₂, —CH(OH)CH₃, —CH(CH₃)₂, —CH₂—CH(CH₃)₂, —CH(CH₃)—C₂H₅, —CH₂—C₆H₅, —CH₂—C₆H₄—OH, —C₂H₄—S—CH₃,
—CH₂—COOH, —CH₂—CONH₂, —C₂H₄—COOH, —C₂H₄—CONH₂, —C₃H₆—NH—C(NH)—NH₂, —C₄H₈—NH₂, 5-imidazolyl or ₂-indolyl, or R³, R⁴, R⁵, R⁶, R⁷, R⁸, R⁹ and R¹⁰ and the neighbouring nitrogen atom together with the atoms to which they are bound form a pyrrolidine ring;
   Z₁ means

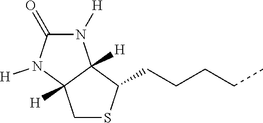

or
—(CH₂)ₒ₁—(O—C₂H₄)ₒ₂—O—(CH₂)ₒ₃-T¹, wherein
o1 is an integer selected from 1, 2, 3, 4, 5 and 6;
o2 is an integer selected from 0, 1, 2, 3, 4, 5, 6, 7, 8, 9 and 10;
o3 is an integer selected from 1, 2, 3, 4, 5 and 6;
Z₂ means —(CH₂)ₒ₄—(O—C₂H₄)ₒ₅—O—(CH₂)ₒ₆-T², wherein
o4 is an integer selected from 1, 2, 3, 4, 5 and 6;
o5 is an integer selected from 0, 1, 2, 3, 4, 5, 6, 7, 8, 9 and 10;
o6 is an integer selected from 1, 2, 3, 4, 5 and 6;
T¹ means —H, —NH₂, —OCH₃, —CO—CH₃, —CO—OCH₃, —CO—NH₂, —NH—CO—CH₃ or

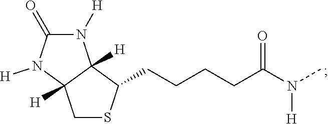

T² means —H, —NH₂, —OCH₃, —CO—CH₃, —CO—OCH₃, —CO—NH₂ or —NH—CO—CH₃;
as well as enantiomers, stereoisomeric forms, mixtures of enantiomers, diastereomers, mixtures of diastereomers, hydrates, solvates, tautomers, racemates of the aforementioned compounds and pharmaceutically acceptable salts thereof.

The peptide residues P₁ and P₂ consist of α-amino acid units Q₁ to Q₈, which are linked to each other via a peptidic amide bond (—CO—NH—). The amino acid units can originate from proteinogenic and non-proteinogenic naturally occurring as well as synthetic amino acids. They can be L-configured and D-configured. Within a peptide residue P₁ or P₂, all amino acids can be exclusively L-configured (all-L), exclusively

D-configured (all-D) or mixed L/D-configured. In the case of a mixed L/D configuration, the configurations can be statistically distributed or alternating.

The peptide residue P₁ is connected to the phosphocholine-containing residue via its C-terminus and linked to the residue R₁ at its N-terminus.

The peptide residue P₂ is linked via its N-terminus to the phosphocholine-containing residue and linked at its C-terminus to the residue R₂.

The inventors have found that the compound of general formula (I) of the present invention efficiently binds CRP in biological liquids and are thus particularly suitable for the treatment of diseases associated with and/or caused by an elevated CRP level. In particular, the compounds described herein are suitable for the treatment and/or prevention of diseases caused by and/or associated with a C-reactive protein blood level of >>5 mg/L.

A particular advantage of the compounds of formula (I) according to the invention lies in their rapid distribution in the bloodstream and in the interstitial spaces, which renders said compounds particularly suitable for the acute treatment of diseases such as infarcts or strokes. This is achieved in particular by the reduced polarity or increased hydrophobicity of the residue U. A particularly favourable polarity of the compound of formula (I) can be achieved with residues U, X and Y if these contain D-amino acids which differ in their physicochemical characteristics and are combined.

Compounds of the general formula (I) whose residues U, X and Y contain ethylene glycol units (PEG) are also advantageous. Ethylene glycol units have a positive influence on polarity with regard to distribution or speed of distribution in the body and also delay proteolytic degradation.

In addition, compounds of the general formula (I) whose residues U, X and Y contain or consist of proline, alanine, and serine (PAS) are advantageous. Peptides consisting of proline, alanine, and serine are particularly stable and thus delay proteolytic degradation. The combination of these three amino acids is known as PASylation.

The compounds of the general formula (I) comprise a 4-aminophenyl phosphocholine which binds to CRP under physiological conditions, so that the concentration of free CRP in the bloodstream decreases. Preferably, the compounds of general formula (I) of the present invention bind monomeric CRP and/or pentameric CRP.

A further aspect of the present invention relates to a pharmaceutical composition comprising the compound of the general formula (I), a pharmaceutically acceptable carrier, an excipient and/or a diluent.

A further aspect of the present invention relates to the use of the compound of the general formula (I) according to the invention and pharmaceutical compositions thereof as a medicament for binding and/or neutralising C-reactive protein in the blood and other body compartments as well as for treating diseases such as tumour diseases, inflammatory diseases, autoimmune diseases, respiratory diseases, metabolic diseases, vascular occlusions, cardiovascular diseases, post-operative conditions and infectious diseases.

In a further embodiment, the compounds according to the invention described herein as well as pharmaceutical compositions thereof can be used in combination with extracorporeal procedures for lowering CRP levels, such as dialysis or apheresis.

In a further embodiment, the compounds according to the invention described herein as well as pharmaceutical compositions thereof can be used in combination with at least one complement blocker for the treatment of diseases associated with and/or caused by an elevated CRP level.

### DESCRIPTION OF THE INVENTION

### Definitions

The term “physiological conditions” as used herein refers to a temperature, a pH, an osmotic pressure, an osmolality, oxidative stress, an electrolyte concentration, a concentration of a small organic molecule such as glucose, lactic acid, pyruvate, nutrient components, other metabolites and the like, a concentration of another molecule such as oxygen, carbonate, phosphate and carbon dioxide, and cell types and nutrient availability that would be considered to be within a normal range at the site of administration or at the tissue or organ at the site of action on a subject.

Preferably, the physiological conditions refer to the conditions prevailing in the human organism of a temperature of 37° C., including pathophysiological boundary conditions such as fever (temperature 37.5° C.-42° C.), a pH of 7.4, including a local variation of 5.0 to 8.5, such as in a wound or a disease, and an osmotic pressure of about 300 mosmol/kg.

The term “biological liquid”, as used herein, refers to aqueous solutions that occur in mammals and preferably humans and contain CRP, such as cerebrospinal fluid, peritoneal fluid, pleural fluid, ascitic fluid, blood, blood plasma, liver extracts and interstitial fluid.

The term “CRP-binding”, as used herein, means that the compounds described herein are capable of reacting with and/or binding to C-reactive protein via their terminal phosphorylcholine group and/or forming a complex with CRP.

The term “neutralisation”, as used herein, refers to the prevention of the pro-inflammatory effect of CRP and the interaction with the humoral and cellular immune system. Neutralisation can thus be achieved by suppressing the binding of CRP to phosphocholine and/or phospholipid components of destroyed endogenous cells, or by preventing complement activation, or by preventing binding to phagocytes.

The term “complement blocker” or “complement inhibitor”, as used herein, refers to drugs that reduce and/or inhibit the activity of individual components of the complement system or the entire complement system. Complement blockers or complement inhibitors therefore bind to plasma proteins such as the C3 and/or the C5 plasma protein at molecularly defined sites and thus prevent complement activation.

The term “post-operative condition”, as used herein, refers to the condition of a patient following a surgical procedure (invasive intervention into the patient's body performed by a surgeon) including vasoconstriction, thromboembolic disease and musculoskeletal injury and post-organ transplant condition.

The term “body compartment”, as used herein, refers to sub-areas of the human body, such as extracellular space, intercellular space, organs, tissue or interstitium. These areas do not necessarily have to be spatially delimitable structures, but can also be constructed only mentally.

The present invention relates to a compound of general formula (I):

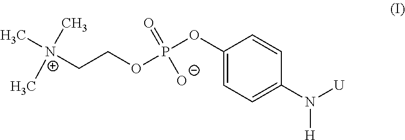

wherein U is selected from —P₁—R₁, —CO—Z₁, —P₂—Z₂ or

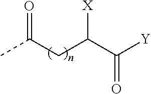

wherein n is an integer selected from 1, 2 and 3;
X represents —NH₂, —NH—P₁—R₁, —NH—CO—Z₁ or —NH—P₁—Z₁;
Y represents —OH, —NH₂, —P₂—R₂, —NH—Z₂ or —P₂—Z₂;
R₁ is selected from —H, —CH₃, —CH₂CH₃, —CO—CH₃ and —CO—CH₂CH₃;
R₂ is selected from —OH, —OCH₃, —OCH₂CH₃, —CH₃, and —NH₂;
P₁ is selected from -Q₁-, -Q₁-Q₂-, -Q₁-Q₂-Q₃- and -Q₁-Q₂-Q₃-Q₄-;
P₂ is selected from −Q₅-, -Q₅-Q₆-, -Q₅-Q₆-Q₇- and -Q₅-Q₆-Q₇-Q₈-;

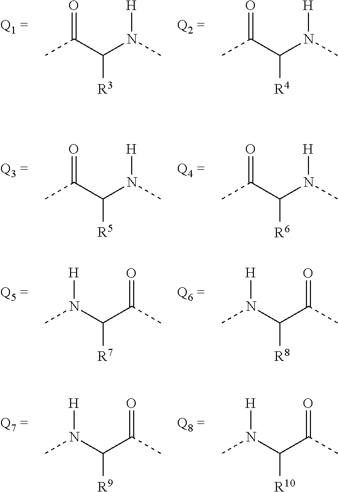

wherein R³, R⁴, R⁵, R⁶, R⁷, R⁸, R⁹ and R¹⁰ represent independently of each other —H, —CH₃, —CH₂OH, —CH₂SH, —COOH, —CONH₂, —CH(OH)CH₃, —CH(CH₃)₂, —CH₂—CH(CH₃)₂, —CH(CH₃)—C₂H₅, —CH₂—C₆H₅, —CH₂—C₆H₄—OH, —C₂H₄—S—CH₃,
—CH₂—COOH, —CH₂—CONH₂, —C₂H₄—COOH, —C₂H₄—CONH₂, —C₃H₆—NH—C(NH)—NH₂, —C₄H₈—NH₂, 5-imidazolyl or 2-indolyl, or R³, R⁴, R⁵, R⁶, R⁷, R⁸, R⁹ and R¹⁰ and the neighbouring nitrogen atom together with the atoms to which they are bound form a pyrrolidine ring;
Z₁ means

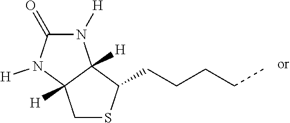

or
μ(CH₂)ₒ₁—(O—C₂H₄)ₒ₂—O—(CH₂)ₒ₃-T¹, wherein
o1 is an integer selected from 1, 2, 3, 4, 5 and 6;
o2 is an integer selected from 0, 1, 2, 3, 4, 5, 6, 7, 8, 9 and 10;
o3 is an integer selected from 1, 2, 3, 4, 5 and 6;
Z₂ means —(CH₂)ₒ₄—(O—C₂H₄)ₒ₅—O—(CH₂)ₒ₆-T², wherein
o4 is an integer selected from 1, 2, 3, 4, 5 and 6;
o5 is an integer selected from 0, 1, 2, 3, 4, 5, 6, 7, 8, 9 and 10;
o6 is an integer selected from 1, 2, 3, 4, 5 and 6;
T¹ means —H, —NH₂, —OCH₃, —CO—CH₃, —CO—OCH₃, —CO—NH₂, —NH—CO—CH₃ or

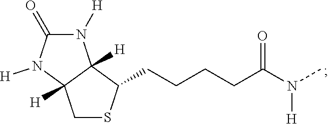

T² means —H, —NH₂, —OCH₃, —CO—CH₃, —CO—OCH₃, —CO—NH₂ or —NH—CO—CH₃;
as well as enantiomers, stereoisomeric forms, mixtures of enantiomers, diastereomers, mixtures of diastereomers, hydrates, solvates, tautomers, racemates of the aforementioned compounds and pharmaceutically acceptable salts thereof.

Preferably, the peptide residues P₁ and P₂ do not consist of more than one amino acid unit, i.e. P₁ is -Q₁- and P₂ is -Q₅-. In a further embodiment, U is represented by —P₁—H, preferably -Q₁-H, wherein P₁ in this case consists of one amino acid unit. In a further embodiment, U is represented by -Q₁-H, wherein Q₁ is a D-configured amino acid unit.

In a further preferred embodiment of the invention, the peptide residues P₁ and P₂ consist of 2 to 4 amino acid units represented by -Q₁-Q₂-, -Q₁-Q₂-Q₃- and -Q₁-Q₂-Q₃-Q₄- or -Q₅-Q₆-, -Q₅-Q₆-Q₇- and -Q₅-Q₆-Q₇-Q₈-.

Preferred are thus compounds of the general formula (Ia)

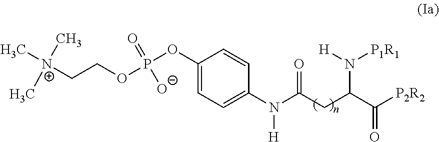

wherein n, P₁, P₂, R₁ and R₂ have the meanings disclosed herein.

In formula (Ia) n is preferably 2.

In formula (Ia) P₁ means preferably -Q₁-Q₂-, -Q₁-Q₂-Q₃- or -Q₁-Q₂-Q₃-Q₄- and P₂ means preferably -Q₅-Q₆-, -Q₅-Q₆-Q₇- or -Q₅-Q₆-Q₇-Q₈-.

R₁ and R₂ have the meanings disclosed herein and preferably R₁ is —H, —CH₃ or —CO—CH₃ and further preferably is —H or —CO—CH₃ and further preferably R₁ is —H and R₂ is preferably-OH, —OCH₃, —OCH₂CH₃ or —NH₂, further preferably is

—OH, —OCH₃ or —NH₂, still further preferably is —OH or —NH₂, and still further preferably R₂ is —NH₂.

The amino acid units Q₁-Q₈ preferably represent proteinogenic amino acid units and further preferably alanine, arginine, asparagine, aspartic acid, glutamine, glutamic acid, glycine, isoleucine, leucine, lysine, methionine, proline, serine, threonine and valine, still further preferably alanine, arginine, asparagine, aspartic acid, glutamine, glutamic acid, lysine, methionine, proline, serine, threonine and valine, still further preferably alanine, arginine, asparagine, glutamine, methionine, proline, serine, threonine and valine, still further preferably alanine, asparagine, glutamine, proline, serine and threonine, further preferably alanine, proline, serine and threonine or for alanine, asparagine, glutamine, proline and serine, and most preferably alanine, proline and serine.

Particularly preferred residues are, for example, —NH-Q₁-Q₂-Q₃-R₁, —NH-Q₁-Q₂-Q₃-Z₁, -Q₅-Q₆-Q₇-R₂ and -Q₅-Q₆-Q₇-Z₂, in which Q₁, Q₂, Q₃, Q₅, Q₆, and Q₇ denote the aforementioned amino acids.

For —NH-Q₁-Q₂-Q₃-R₁ and -Q₅-Q₆-Q₇-R₂ with Q₁=Q₇=serine and Q₂=Q₆=alanine and Q₃=Q₅=proline, the following residues result:

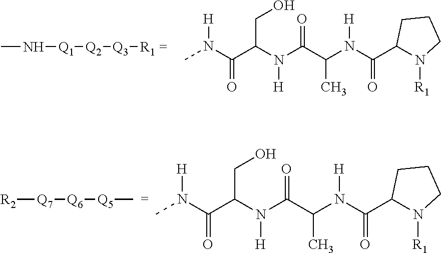

A further aspect of the present invention relates to a compound of general formula (II)

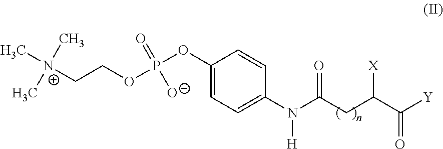

wherein, X and Y have the meanings and preferred meanings defined herein, preferably with n=2.

In all general formulae disclosed herein, n is preferably an integer selected from 2 and 3. Particularly preferred is when n=2.

In a preferred embodiment of the compound of the general formula (II), X is a primary amino group and Y is —OH or —NH₂. In a further preferred embodiment of the compound of the general formula (II), n is 2, Y is —OH and X is —NH₂.

Furthermore, the compounds of the general formula (II) are preferred, in which n=2,
X represents —NH—P₁—R₁,
Y represents —P₂—R₂,
P₁ is selected from -Q₁-Q₂-, -Q₁-Q₂-Q₃- and -Q₁-Q₂-Q₃-Q₄-;
P₂ is selected from -Q₅-Q₆-, -Q₅-Q₆-Q₇- and -Q₅-Q₆-Q₇-Q₈-;
and R₁, R₂ as well as Q₁-Q₅ have the meanings and preferred meanings as defined herein, particularly as described on the previous page.

Preferred are also compounds structure of general formula (II), in which
n is 2,
Y represents —OH or —NH₂,
X represents —NH₂ or —NH—CO—Z₁, wherein
   Z₁ is

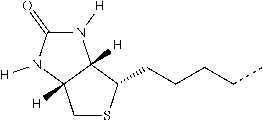

or —(CH₂)ₒ₁—(O—C₂H₄)ₒ₂—O—(CH₂)ₒ₃-T
o1 is 1 or 2,
o2 is an integer selected from 1, 2, 3, 4 and 5;
o3 is 1 or 2 and
   T¹ is —NH—CO—CH₃,

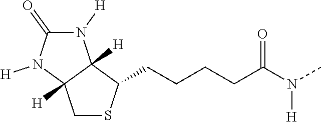

### or —NH₂.

In a preferred embodiment of the compound of the general formula (II), X is —NH—CO—Z₁ and Y is —OH or —NH₂, wherein Z₁ represents —(CH₂)ₒ₁—(O—C₂H₄)ₒ₂—O—(CH₂)ₒ₃-T¹ and o1, o2, o3, and T¹ have the meanings as defined herein. Preferably, o1 and o3 are independently of each other selected from 1 and 2 and o2 is an integer selected from 1, 2, 3, 4 and 5. Even more preferably, o1 is 1, o2 is 1, O3 is 2 and T¹ is —NH—CO—CH₃.

Preferably, in all residues —(CH₂)ₒ₁—(O—C₂H₄)ₒ₂—O—(CH₂)ₒ₃-T¹ as disclosed herein, o1 and o3 are independently of each other selected from 1 and 2. Likewise, o2 is preferably an integer selected from 1, 2, 3, 4 and 5 Further preferably, in all residues —(CH₂)ₒ₁—(O—C₂H₄)ₒ₂—O—(CH₂)ₒ₃-T¹ as disclosed herein, o1 and o3 are independently of each other selected from 1 and 2 and o2 is an integer selected from 1, 2, 3, 4 and 5, preferably selected from 1, 2 and 3.

In a preferred embodiment of the compound of the general formula (II), X is —NH₂ and Y is —NH—Z₂, wherein Z₂ represents —(CH₂)ₒ₄—(O—C₂H₄)ₒ₅—O—(CH₂)ₒ₆-T² and o4, o5, o6 and T² have the meanings as defined herein. Preferably, o4 and o6 are independently of each other selected from 1 and 2 and o5 is an integer selected from 1, 2, 3, 4 and 5. Even more preferably, o4 is 2, o6 is 1, o5 is 1 and T² is —CO—NH₂.

In a preferred embodiment of the compound of the general formula (II), X is —NH—CO—Z₁ and Y is —NH—Z₂, wherein Z₁ represents —(CH₂)ₒ₁—(O—C₂H₄)ₒ₂—O—(CH₂)ₒ₃-T¹ and Z₂ represents —(CH₂)ₒ₄—(O—C₂H₄)ₒ₅—O—(CH₂)ₒ₆-T²; o1-o6 as well as T¹ and T² have the meanings given herein and preferably o1, o3, o4 and o6 are independently of each other selected from 1, 2 and 3, preferably from 1 and 2; and o2 and o5 independently of each other selected from 1, 2, 3, 4 and 5, preferably from 1, 2, 3 and 4 and further preferably from 1, 2 and 3; and T¹ represents —H, —OCH₃, —CO—NH₂, —NH—CO—CH₃ or —NH₂, preferably —H, —OCH₃ or —NH₂ and further preferably —H or —OCH₃; and T² represents —H, —OCH₃, —CO—NH₂, —NH—CO—CH₃ or —NH₂, preferably —H, —OCH₃ or —NH₂ and further preferably-H or —OCH₃.

Particularly preferred are compounds of general formula (II), wherein
Y represents —OH or —NH₂,
X represents —NH₂ or —NH—CO—Z₁, wherein
Z₁ is —(CH₂)ₒ₁—(O—C₂H₄)ₒ₂—O—(CH₂)ₒ₃-T¹, with
o1 being 1 or 2,
o2 being an integer selected from 1, 2, 3, 4 and 5,
o3 being 1 or 2 and with
T¹ being —NH₂.

In a further preferred embodiment of the compound of the general formula (II), n equals 2,
X represents —NH₂ or —NH—P₁—R₁,
Y represents —OH, —NH₂ or —P₂—R₂,
P₁ and P₂ represent independently of each other -AS₁-, -AS₁-AS₂- or -AS₁-AS₂-AS₃- and
AS₁, AS₂ and AS₃ represent for each occurrence independently of each other amino acids of the following structure:

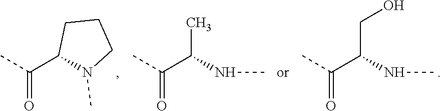

wherein R₁ and R₂ have the meanings and preferred meanings as defined herein.

Preferably, P₁ and P₂ are -AS₁-AS₂-AS₃-. Even more preferably, P₁ is -AS₁-AS₂-AS₃- and P₂ is -AS₄-AS₅-AS₆-, R₁ represents —CO—CH₃ and R₂ is —H, wherein AS₁-AS₆ are the following amino acids:

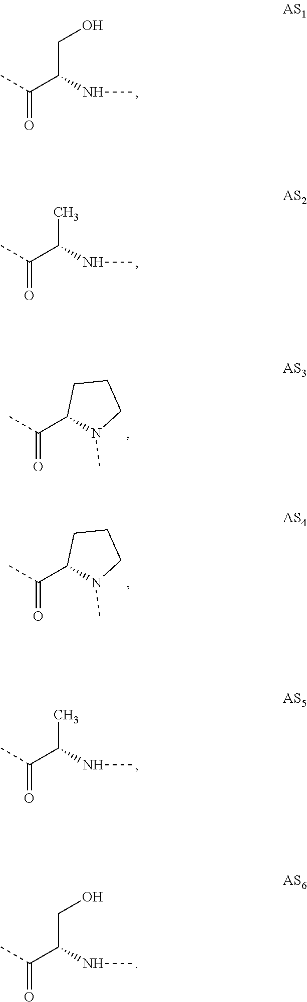

In a particularly preferred embodiment, the compound according to the invention has the structure of the general formula (IIa)

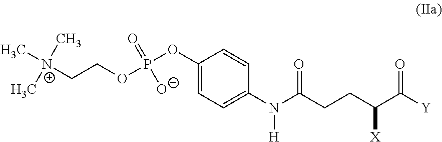

wherein X and Y have the meanings and preferred meanings as defined herein.

In a preferred embodiment of the compound of the general formula (IIa), X is a primary amino group and Y is —OH or —NH₂.

Further preferred are compounds of general formula (IIa), in which X represents —NH—CO—Z₁ and Y represents —OH or —NH₂, wherein Z₁ means —(CH₂)ₒ₁—(O—C₂H₄)ₒ₂—O—(CH₂)ₒ₃-T¹ and o1, o2, o3 and T¹ have the meanings and preferred meanings as defined herein. Preferably, o1 and o3 are independently of each other an integer selected from 1 and 2; and o2 is an integer selected from 1, 2, 3, 4 and 5, preferably 1, 2, 3 and 4, further preferably 1, 2, and 3. Even more preferably, o1 is 1, o2 is 1, o3 is 2 and T¹ is —NH—CO—CH₃.

In a preferred embodiment of the compound of the general formula (IIa), X is —NH₂ and Y is —NH—Z₂, wherein Z₂ represents —(CH₂)ₒ₄—(O—C₂H₄)ₒ₅—O—(CH₂)ₒ₆-T² and o4, o5, o6 and T² have the meanings and preferred meanings as defined herein. Preferably, o4 and o6 are independently of each other an integer selected from 1 and 2; and o5 is an integer selected from 1, 2, 3, 4 and 5, preferably 1, 2, 3 and 4, further preferably 1, 2, and 3. Even more preferably, o4 is 2, o5 is 1, o6 is 1 and T² is —CO—NH₂.

Further preferred are compounds of general formula (IIa), in which X means —NH—CO—Z₁ and Y means —NH—Z₂, wherein Z₁ represents —(CH₂)ₒ₁—(O—C₂H₄)ₒ₂—O—(CH₂)ₒ₃-T¹ and Z₂ represents —(CH₂)ₒ₄—(O—C₂H₄)ₒ₅—O—(CH₂)ₒ₆-T²; o1-o6 as well as T¹ and T² have the meanings and preferred meanings as defined herein and preferably o1, o3, o4 and o6 are independently of each other selected from 1, 2 and 3, preferably from 1 and 2; and o2 and o5 are independently of each other selected from 1, 2, 3, 4 and 5, preferably from 1, 2, 3 and 4 and further preferably from 1, 2 and 3; and T¹ represents —H, —OCH₃, —CO—NH₂, —NH—CO—CH₃ or —NH₂, preferably —H, —OCH₃ or —NH₂ und further preferably —H
or —OCH₃; and T² represents —H, —OCH₃, —CO—NH₂, —NH—CO—CH₃ or —NH₂, preferably —H, —OCH₃ or —NH₂ and further preferably —H or —OCH₃.

Even more preferably, o1 is 1, o2 is 1, o3 is 2, T¹ is —NH—CO—CH₃, o4 is 2, o5 is 1, o6 is 1 and T² is —CO—NH₂.

Particular preferred are compounds of general formula (IIa), wherein
Y represents —OH or —NH₂,
X represents —NH₂ or —NH—CO—Z₁, wherein
Z₁ is —(CH₂)ₒ₁—(O—C₂H₄)ₒ₂—O—(CH₂)ₒ₃-T¹, with
o1 being 1 or 2,
o2 being an integer selected from 1, 2, 3, 4 and 5;
o3 being 1 or 2; and with
T¹ being —NH₂.

In a further preferred embodiment of the compound of general formula (IIa), n equals 2,
X represents —NH₂ or —NH—P₁—R₁,
Y represents —OH, —NH₂ or —P₂—R₂,
P₁ and P₂ represent independently of each other -AS₁-, -AS₁-AS₂- or -AS₁-AS₂-AS₃- and
AS₁, AS₂ and AS₃ are for each occurrence independently of each other amino acids of the following structure:

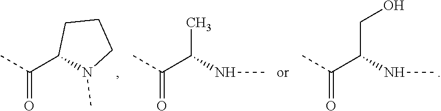

wherein R₁ and R₂ have the meanings and preferred meanings as defined herein.

Preferably, P₁ and P₂ are -AS₁-AS₂-AS₃-. Even more preferably, P₁ is -AS₁-AS₂-AS₃- and P₂ is -AS₄-AS₅-AS₆-, R₁ represents —CO—CH₃ and R₂ is —H, wherein AS₁-AS₆ are the following amino acids:

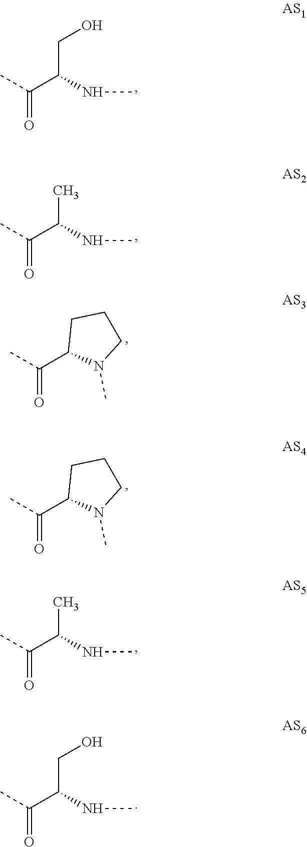

In a particularly preferred embodiment, the compound according to the invention has the structure of the general formula (IIb)

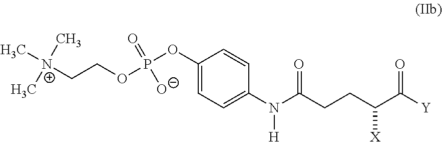

wherein X and Y have the meanings and preferred meanings as defined herein.

In a preferred embodiment of the compound of the general formula (IIb), X is a primary amino group and Y is —OH or —NH₂.

In a preferred embodiment of the compound of the general formula (IIb) X is —NH—CO—Z₁ and Y is —OH or —NH₂, wherein Z₁ means —(CH₂)ₒ₁—(O—C₂H₄)ₒ₂—O—(CH₂)ₒ₃-T¹ and o1, o2, o3 and T¹ have the meanings and preferred meanings as defined herein. Preferably, o1 and o3 are independently of each other an integer selected 1 and 2 and o2 is an integer selected from 1, 2, 3, 4 and 5 preferably 1, 2, 3 and 4, further preferably 1, 2, and 3. Even more preferably, o1 is 1, o2 is 1, o3 is 2 and T¹ is —NH—CO—CH₃.

In a preferred embodiment of the compound of the general formula (IIb), X is —NH₂ and Y is —NH—Z₂, wherein Z₂ represents —(CH₂)ₒ₄—(O—C₂H₄)ₒ₅—O—(CH₂)ₒ₆-T² and o4, o5, o6 and T² have the meanings and preferred meanings defined herein.

Preferably, o4 and o6 are independently of each other an integer selected 1 and 2; and o5 is an integer selected from 1, 2, 3, 4 and 5, preferably 1, 2, 3 and 4, further preferably 1, 2, and 3. Even more preferably, o4 is 2, o5 is 1, o6 is 1 and T² is —CO—NH₂.

In a further preferred embodiment of the compound of the general formula (IIb), X is —NH—CO—Z₁ and Y means —NH—Z₂, wherein Z₁ represents —(CH₂)ₒ₁—(O—C₂H₄)ₒ₂—O—(CH₂)ₒ₃-T¹ and Z₂ represents —(CH₂)ₒ₄—(O—C₂H₄)ₒ₅—O—(CH₂)ₒ₆-T²; o1-o6 as well as T¹ and T² have the meanings and preferred meanings as defined herein and preferably o1, o3, o4 and o6 are independently of each other selected from 1, 2 and 3, preferably from 1 and 2; and o2 and o5 are independently of each other selected from 1, 2, 3, 4 and 5, preferably from 1, 2, 3 and 4 and further preferably from 1, 2 and 3; and T¹ represents —H, —OCH₃, —CO—NH₂, —NH—CO—CH₃ or —NH₂, preferably —H, —OCH₃ or —NH₂ und further preferably —H or —OCH₃; and T² represents —H, —OCH₃, —CO—NH₂, —NH—CO—CH₃ or —NH₂, preferably —H, —OCH₃ or —NH₂ and further preferably —H or —OCH₃.

Even more preferably, o1 is 1, o2 is 1, o3 is 2, T¹ is —NH—CO—CH₃, o4 is 2, o5 is 1, o6 is 1 and T² is —CO—NH₂.

Particular preferred are compounds of general formula (IIb), wherein
Y represents —OH or —NH₂,
X represents —NH₂ or —NH—CO—Z₁, wherein
Z₁ is —(CH₂)ₒ₁—(O—C₂H₄)ₒ₂—O—(CH₂)ₒ₃-T¹, with
o1 being 1 or 2,
o2 being an integer selected from 1, 2, 3, 4 and 5;
o3 being 1 or 2; and with
T¹ being —NH₂.

In a further preferred embodiment of the compound of general formula (IIb), n equals 2,
X represents —NH₂ or —NH—P₁—R₁,
Y represents —OH, —NH₂ or —P₂—R₂,
P₁ and P₂ represent independently of each other -AS₁-, -AS₁-AS₂- or -AS₁-AS₂-AS₃- and
AS₁, AS₂ and AS₃ are for each occurrence independently of each other amino acids of the following structure:

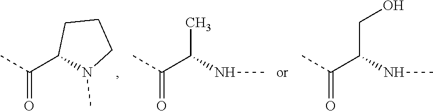

wherein R₁ and R₂ have the meanings and preferred meanings defined herein.

Preferably, P₁ and P₂ are -AS₁-AS₂-AS₃-. Even more preferably, P₁ is -AS₁-AS₂-AS₃-P₂ is -AS₄-AS₅-AS₆-, R₁ represents —CO—CH₃ and R₂ is —H, wherein AS₁-AS₆ are the following amino acids:

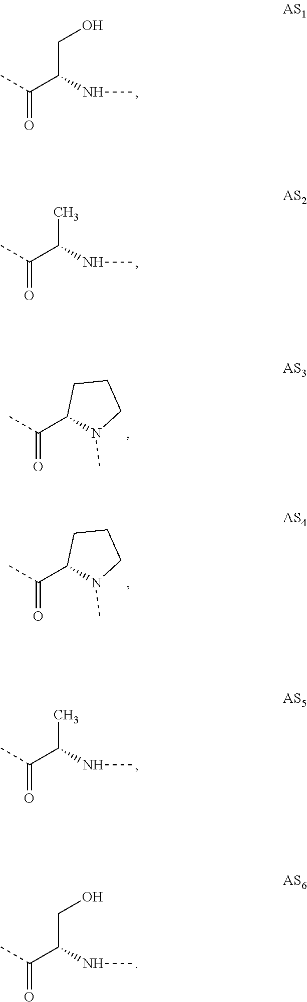

In a particularly preferred embodiment, the compound according to the invention has the structure of the general formula (III)

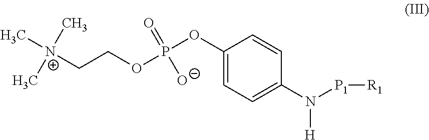

wherein P₁ represents -Q₁-, -Q₁-Q₂-, -Q₁-Q₂-Q₃- or -Q₁-Q₂-Q₃-Q₄-, with

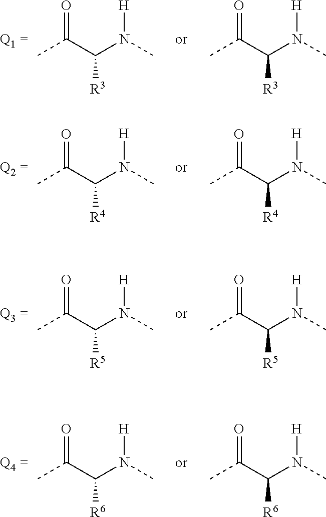

R₁ is selected from —H, —CH₃, —CH₂CH₃, —CO—CH₃ and —CO—CH₂CH₃;
R₃, R₄, R₅, and R₆ mean independently of each other —H, —CH₃, —CH₂OH, —CH₂SH, —COOH, —CONH₂, —CH(OH)CH₃, —CH(CH₃)₂, —CH₂—CH(CH₃)₂, —CH(CH₃)—C₂H₅, —CH₂—C₆H₅, —CH₂—C₆H₄—OH, —C₂H₄—S—CH₃, —CH₂—COOH, —CH₂—CONH₂, —C₂H₄—COOH, —C₂H₄—CONH₂, —C₃H₆—NH—C(NH)—NH₂, —C₄H₈—NH₂, 5-imidazolyl or 2-indolyl, or R₃, R₄, R₅, and R₆ and the neighbouring nitrogen atom together with the atoms to which they are bound form a pyrrolidine ring (like in the amino acid proline);
as well as stereoisomeric forms, mixtures of enantiomers, diastereomers, mixtures of diastereomers, hydrates, solvates, tautomers, racemates of the aforementioned compounds and pharmaceutically acceptable salts thereof.

Preferably, P₁=-Q₁- and R₁=—H. Also preferably, R₃ is selected from —H, —CH₂—COOH, —CH₂—CONH₂, —C₂H₄—COOH, —C₂H₄—CONH₂ or —C₂H₄—S—CH₃.

In a particularly preferred embodiment, the compound according to the invention has the structure of the general formula (IIIa)

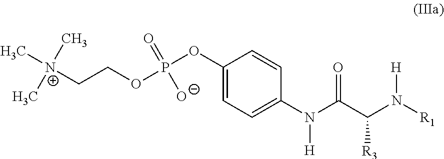

wherein R₃ represents —H, —CH₃, —CH₂OH, —CH₂SH, —COOH, —CONH₂, —CH(OH)CH₃, —CH(CH₃)₂, —CH₂—CH(CH₃)₂, —CH(CH₃)—C₂H₅, —CH₂—C₆H₅, —CH₂—C₆H₄—OH, —C₂H₄—S—CH₃, —CH₂—COOH, —CH₂—CONH₂, —C₂H₄—COOH, —C₂H₄—CONH₂, —C₃H₆—NH—C(NH)—NH₂, —C₄H₈—NH₂, 5-imidazolyl or 2-indolyl, or
R₃ and the neighbouring nitrogen atom together with the atoms to which they are bound form a pyrrolidine ring (like in the amino acid proline) and R₁ has the meanings and preferred meanings as defined herein.

Preferably, R₁=—H and/or also preferably, R₃ is selected from —H, —CH₂—COOH, —CH₂—CONH₂, —C₂H₄—COOH, —C₂H₄—CONH₂, —C₂H₄—S—CH₃ or R₃ and the neighbouring nitrogen atom together with the atoms to which they are bound form a pyrrolidine ring.

In a particularly preferred embodiment, the compound according to the invention has the structure of the general formula (IIIb)

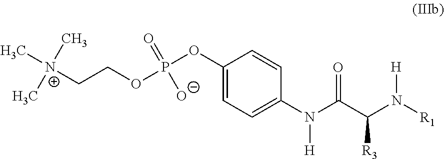

wherein R₃ represents —H, —CH₃, —CH₂OH, —CH₂SH, —COOH, —CONH₂, —CH(OH)CH₃, —CH(CH₃)₂, —CH₂—CH(CH₃)₂, —CH(CH₃)—C₂H₅, —CH₂—C₆H₅, —CH₂—C₆H₄—OH, —C₂H₄—S—CH₃, —CH₂—COOH, —CH₂—CONH₂, —C₂H₄—COOH, —C₂H₄—CONH₂, —C₃H₆—NH—C(NH)—NH₂, —C₄H₈—NH₂, 5-imidazolyl or 2-indolyl, or
R₃ and the neighbouring nitrogen atom together with the atoms to which they are bound form a pyrrolidine ring (like in the amino acid proline)
and R₁ has the meanings and preferred meanings as defined herein.

Preferably, R₁=—H and/or also preferably, R₃ is selected from —H, —CH₂—COOH, —CH₂—CONH₂, —C₂H₄—COOH, —C₂H₄—CONH₂, —C₂H₄—S—CH₃ or R₃ and the neighbouring nitrogen atom together with the atoms to which they are bound form a pyrrolidine ring.

In a particularly preferred embodiment, the compound according to the invention has the structure of the general formula (IIIc)

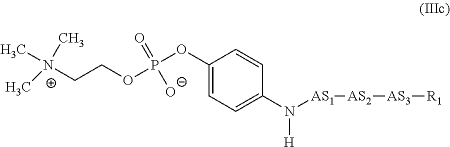

wherein AS₁, AS₂ and AS₃ represent independently of each other the following structure:

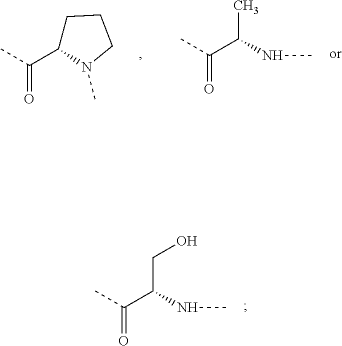

and R₁ has the meanings and preferred meanings as defined herein.

Preferably, R₁=—H.

It is preferred if

AS₁ is

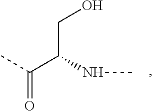

### AS₂ is

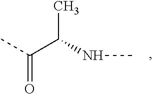

### and AS₃ is

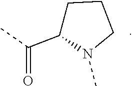

It is also preferred if
AS₁

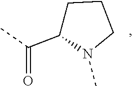

### AS₂ is

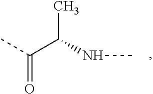

### and AS₃ is

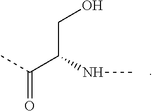

In a particularly preferred embodiment, the compound according to the invention has the structure of the general formula (IV)

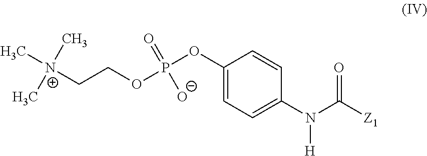

wherein Z₁ represents —(CH₂)ₒ₁—(O—C₂H₄)ₒ₂—O—(CH₂)ₒ₃-T¹;
o1 is an integer selected from 1, 2, 3, 4, 5 and 6, preferably from 1, 2, 3 and 4, further preferably from 1, 2, and 3 and particularly preferably from 1 and 2;
o2 is an integer selected from 1, 2, 3, 4, 5, 6, 7, 8, 9 and 10, preferably from 1, 2, 3, 4, 5, 6, 7 and 8, further preferably from 1, 2, 3, 4, 5 and 6 and particularly preferably from 1, 2, 3 and 4;
o3 is an integer selected from 1, 2, 3, 4, 5 and 6, preferably from 1, 2, 3 and 4, further preferably from 1, 2, and 3 and particularly preferably from 1 and 2;
T¹ represents —H, —OCH₃, —CO—NH₂, —NH—CO—CH₃ or —NH₂, preferably —H, —OCH₃ or —NH₂ and further preferably —NH₂ or —OCH₃.

Preferably, o1 is 1 or 2,
o2 is an integer selected from 1, 2, 3, 4 and 5,
o3 is 1 or 2, and T¹=—NH₂.

Particularly preferred are the compounds 1, 3, 4 und 5.

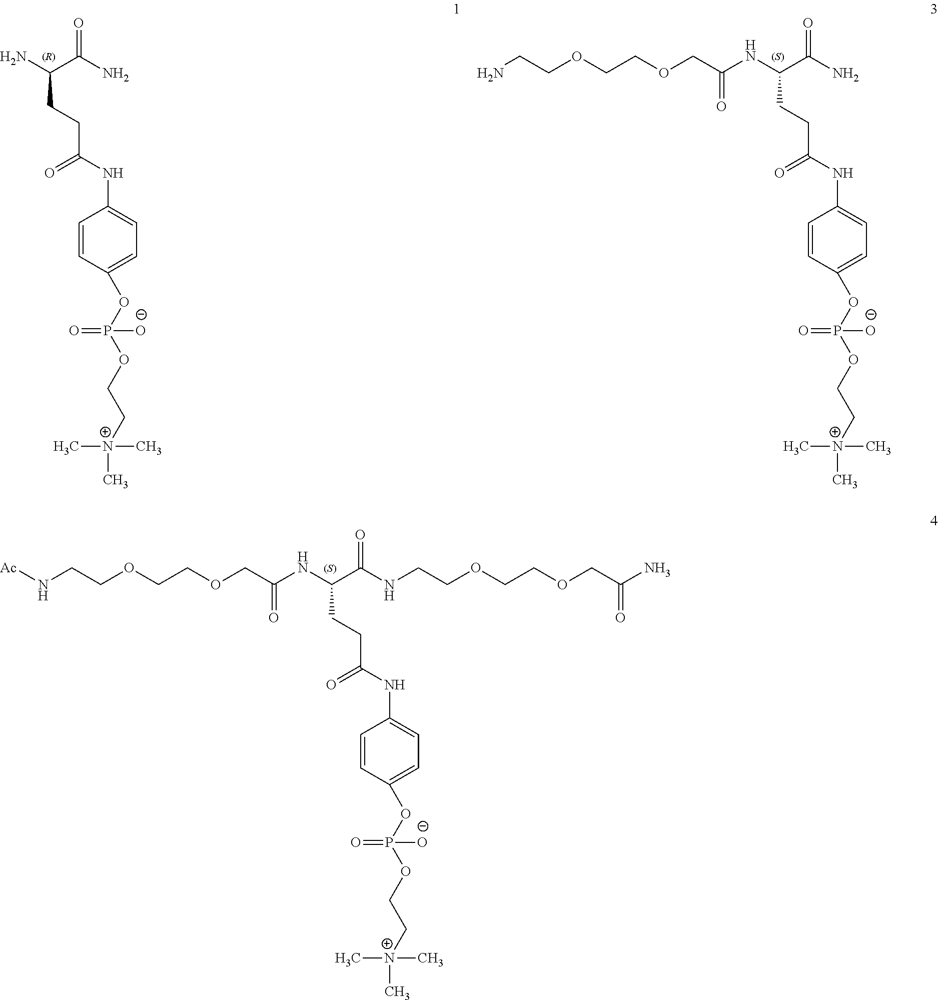

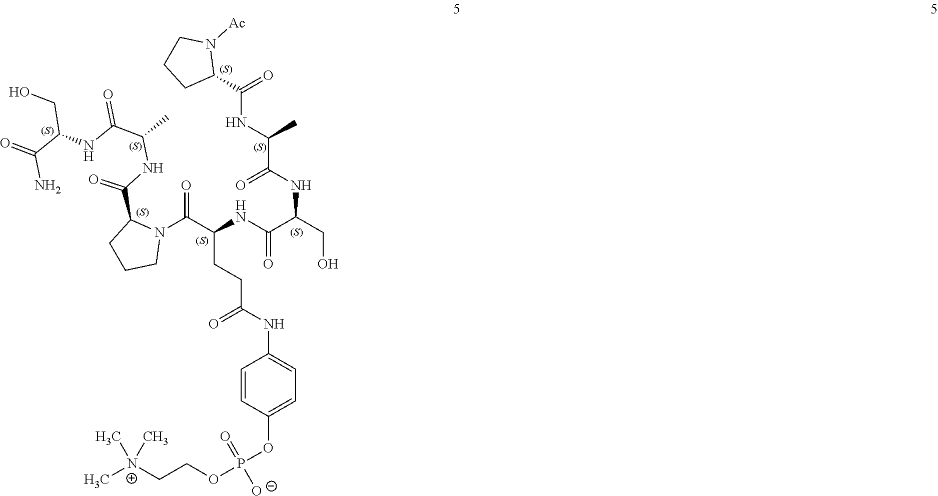

Surprisingly, the inventor has found that the compounds of general formulae (I), (Ia), (II), (IIa), (IIb), (III), (IIIa), (IIIb), (IIIc) and (IV) bind efficiently to CRP and thereby reduce inflammation-induced cell, tissue and vascular destruction. The compounds of the general formulae (I), (Ia), (II), (IIa), (IIb), (III), (IIIa), (IIIb), (IIIc) and (IV) described herein are capable of binding to the CRP in such a way that subsequent complexation with the complement protein C1q no longer takes place or only takes place to a lesser extent. This stops, reduces or even suspends the pro-oedematous and pronecrotic effect of CRP in diseases such as myocardial infarction and stroke. Ultimately, the spread of damage and formation of scars in ischaemic tissue can be stopped and infarct scars after a heart attack can be reduced or prevented altogether.

The compounds of the general formulae (I), (Ia), (II), (IIa), (IIb), (III), (IIIa), (IIIb), (IIIc) and (IV) described herein can be synthesized from 4-aminophenylphosphocholine and amino acid building blocks using the methods known from the prior art for synthesising peptide conjugates. Amino acids have a large number of functional groups known to the skilled person, via which the 4-aminophenylphosphocholine (APPC) can be covalently bound. Preferably, the APPC is bound via an α-carboxy group or a carboxy group of a glutamate or aspartate residue. The amino acids can be used with the established protecting group strategies, such as Fmoc or Boc protecting groups.

In particular, peptide coupling reagents such as carbodiimides (including dicyclohexyl carbodiimide (DCC), diisopropyl carbodiimide (DIC) and 1-ethyl-3-(3-dimethylaminopropyl)carbodiimide (EDC)), phosphonium salts (including BOP reagent, PyBOP, PyBrOP and PyOxim), immonium salts (including BOMI and BDMP), aminium salts (including HBTU, TBTU, HATU, HCTU and TATU), uronium salts (including TNTU, TPTU, TOTU, TDBTU, COMU, COMBU, TOMBU and TSTU), imidazolium salts (including CBMIT, BOI, CIP, CIB and CMBI) or carbonyl diimidazole are suitable for binding the APPC to the peptidic residue U. The inventor has found that the compounds of general formulae (I), (Ia), (II), (IIa), (IIb), (III), (IIIa), (IIIb), (IIIc) and (IV) described herein efficiently bind CRP in the bloodstream and are thus particularly suitable for the treatment of diseases associated with and/or caused by an elevated CRP level. An elevated CRP level herein means a C-reactive protein blood level of preferably >5 mg/L, more preferably of >10 mg/L, more preferably of >15 mg/L, more preferably of >20 mg/L, more preferably of >25 mg/L, more preferably of >30 mg/L, more preferably of >50 mg/L, more preferably of >75 mg/L, more preferably of >100 mg/L, more preferably of >150 mg/L and most preferably of >200 mg/L at any point in time of the disease.

Thus, a further aspect of the present invention lies in the use of the compounds of general formulae (I), (Ia), (II), (IIa), (IIb), (III), (IIIa), (IIIb), (IIIc) and (IV) n the treatment and/or prevention of diseases caused by and/or associated with a CRP blood level of >20 mg/L,
wherein U is —H, —P₁—R₁, —CO—Z₁, —P₂—Z₂ or

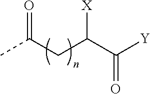

no and n, X, Y, P₁, P₂, R₁, R₂, R₃, AS₁, AS₂, AS₃ and Z₁ have the meanings and preferred meanings as described herein.

However, atherosclerosis is not one of the diseases associated with and/or caused by an elevated CRP level. In atherosclerosis, an elevated CRP level of >20 mg/L in the blood is not regularly observed.

Thus, the present invention relates to the compounds of general formula (I) for use in the treatment and/or prevention of diseases caused and/or associated by a C-reactive protein blood level of >20 mg/L, wherein the disease is not atherosclerosis
wherein U is —H, —P₁—R₁, —CO—Z₁, —P₂—Z₂ or

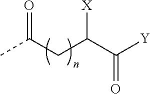

and n, X, Y, P₁, P₂, R₁, R₂, R₃, AS₁, AS₂, AS₃ and Z₁ have the meanings and preferred meanings as described herein.

The diseases associated and/or caused by an elevated CRP level are preferably acute diseases in which an elevated CRP level (as described herein) is observable in the patient within 1 to 5 days. Preferably, these diseases are associated with and/or caused by an increase in the CRP level of >20 mg/L within 1 to 5 days. Preferably, these diseases are associated with and/or caused by an increase in the CRP level of >20 mg/L within 1 to 4 days. Preferably, these diseases are associated with and/or caused by an increase in the CRP level of >20 mg/L within 1 to 3 days. Preferably, these diseases are associated with and/or caused by an increase in the CRP level of >20 mg/L within 1 to 2 days.

Thus, the present invention relates to the compounds of general formulae (I), (Ia), (II), (IIa), (IIb), (III), (IIIa), (IIIb), (IIIc) and (IV) for use in the treatment and/or prevention of diseases caused and/or associated with an acute increase in CRP blood level of >20 mg/L
wherein U is —H, —P₁—R₁, —CO—Z₁, —P₂—Z₂ or

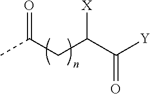

and n, X, Y, P₁, P₂, R₁, R₂, R₃, AS₁, AS₂, AS₃ and Z₁ have the meanings and preferred meanings as described herein.

Diseases that are associated with and/or caused by an elevated CRP level include tumour diseases, inflammatory diseases, metabolic diseases, autoimmune diseases, respiratory diseases, cardiovascular diseases, post-operative conditions and infectious diseases.

Thus, the present invention relates to the compounds of general formulae (I), (Ia), (II), (IIa), (IIb), (III), (IIIa), (IIIb), (IIIc) and (IV) for use in the treatment and/or prevention of diseases selected from tumour diseases, inflammatory diseases, metabolic diseases, autoimmune diseases, respiratory diseases, cardiovascular diseases, post-operative conditions and infectious diseases
wherein U is —H, —P₁—R₁, —CO—Z₁, —P₂—Z₂ or

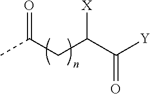

and n, X, Y, P₁, P₂, R₁, R₂, R₃, AS₁, AS₂, AS₃ and Z₁ have the meanings and preferred meanings described herein.

Preferably, the tumour diseases are diseases selected from adenocarcinoma, choroidal melanoma, acute leukaemia, acoustic neurinoma, ampullary carcinoma, anal carcinoma, astrocytoma, basal cell carcinoma, pancreatic cancer, desmoid tumour, bladder cancer, bronchial carcinoma, breast cancer, Burkitt's lymphoma, corpus cancer, CUP syndrome (carcinoma of unknown origin), bowel cancer, small intestine cancer, small intestine tumours, ovarian cancer, endometrial carcinoma, ependymoma, epithelial forms of cancer, Ewing tumours, gastrointestinal tumours, stomach cancer, gallbladder cancer, gallbladder carcinomas, uterine cancer, cervical cancer, cervix, glioblastomas, gynaecological tumours, ear, nose and throat tumours, haematological neoplasia, hairy cell leukaemia, urethral cancer, skin cancer, skin and testicular cancer, brain tumours (gliomas), brain metastases, testicular cancer, pituitary tumours, carcinoids, Kaposi's sarcoma, laryngeal cancer, germ cell tumours, bone cancer, colorectal cancer, head and neck tumours (tumours of the ear, nose and throat), colon cancer, craniopharyngiomas, oral cancer (cancer of the mouth and lips), cancer of the central nervous system, liver cancer, liver metastases, leukaemia, eyelid tumours, lung cancer, lymph node cancer (Hodgkin's/non-Hodgkin's), lymphomas, stomach cancer, malignant melanoma, malignant neoplasia, malignant tumours of the gastrointestinal tract, breast carcinoma, rectal cancer, medulloblastoma, melanoma, meningioma, Hodgkin's disease, mycosis fungoides, myositis, nasal cancer, neurinoma, neuroblastoma, kidney cancer, renal cell carcinoma, non-Hodgkin's lymphoma, oligodendroglioma, oesophageal carcinoma, osteolytic carcinoma and osteoplastic carcinoma, osteosarcoma, ovarian carcinoma, pancreatic carcinoma, penile cancer, plasmocytoma, prostate cancer, throat cancer, rectal carcinoma, retinoblastoma, vaginal cancer, thyroid carcinoma, Schneeberger's disease, oesophageal cancer, spinalioma, T-cell lymphoma (mycosis fungoides), thymoma, tubal carcinoma, eye tumours, urethral cancer, urological tumours, urothelial carcinoma, vulvar cancer, warts, soft tissue tumours, soft tissue sarcoma, Wilms' tumour, cervical carcinoma and tongue cancer.

The present invention also relates to the compounds of general formulae (I), (Ia), (II), (IIa), (IIb), (III), (IIIa), (IIIb), (IIIc) and (IV) for use in the treatment and/or prevention of autoimmune diseases selected from asthma, diabetes, rheumatic diseases, AIDS, rejection of transplanted organs and tissues, rhinitis, chronic obstructive pulmonary disease, osteoporosis, ulcerative colitis, sinusitis, lupus erythematosus, recurrent infections, atopic dermatitis/eczema and occupational allergies, anaphylaxis, manifestations of allergic diseases, primary immunodeficiency, antibody deficiency states, cell-mediated immunodeficiency, severe combined immunodeficiency, DiGeorge syndrome, hyper-IgE syndrome, Wiskott-Aldrich syndrome, ataxia teleangiectasia, immune-mediated forms of cancer, leucocyte defects, autoimmune diseases, systemic lupus erythematosus, rheumatoid arthritis (RA), multiple sclerosis (MS), immune-mediated or type 1 diabetes mellitus, immune-mediated glomerulonephritis, scleroderma, pernicious anaemia, alopecia, pemphigus, pemphigus vulgaris, myasthenia gravis, inflammatory bowel disease, Crohn's disease, psoriasis, autoimmune thyroid disease, Hashimoto's thyroiditis, dermatomyositis, Goodpasture's syndrome, myasthenia gravis pseudoparalytica, ophthalmia sympathica, phacogenic uveitis, chronic aggressive hepatitis, primary biliary cirrhosis, autoimmune haemolytic anaemia and Werlhof's disease
wherein U is —H, —P₁—R₁, —CO—Z₁, —P₂—Z₂ or

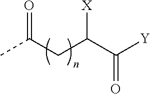

and n, X, Y, P₁, P₂, R₁, R₂, R₃, AS₁, AS₂, AS₃ and Z₁ have the meanings and preferred meanings as described herein.

Preferably, the compounds of the general formulae (I), (Ia), (II), (IIa), (IIb), (III), (IIIa), (IIIb), (IIIc) and (IV) are used for the treatment and/or prevention of infectious diseases selected from AIDS, alveolar echinococcosis (AHD, echinococcosis), amoebiasis (Entamoeba histolytica infection), angiostrongylus infection, anisakiasis, anthrax, babesiosis (Babesia infection), balantidium infection (balantidiosis), Baylisascaris infection (raccoon roundworm), Bilharzia (schistosomiasis), Blastocystis hominis infection (blastomycosis), borreliosis, botulism, Brainerd's diarrhoea, brucellosis, BSE (bovine spongiform encephalopathy), candidiasis, Capillariasis (Capillaria infection), CFS (chronic fatigue syndrome), Chagas disease (American trypanosomiasis), chickenpox (Varicella zoster virus), Chlamydia pneumoniae infection, cholera, chronic fatigue syndrome, CJD (Creutzfeldt-Jakob disease), clonorchiasis (clonorchis infection), LMC (larva migrans cutanea, hookworm infection), coccidioidomycosis, conjunctivitis, coxsackievirus A16 (hand, foot and mouth disease), cryptococcosis, Cryptosporidium infection (cryptosporidiosis), Culex mosquito (vector of West Nile virus), Larva migrans cutanea (LMC), Cyclosporosis (Cyclospora infection), Cysticercosis (neurocysticercosis), Cytomegalovirus infection, Dengue/dengue fever, Dipylidium infection (dog and cat flea tapeworm), Ebola virus haemorrhagic fever, echinococcosis (alveolar echinococcosis), encephalitis, Entamoeba coli infection, Entamoeba dispar infection, Entamoeba hartmanni infection, Entamoeba histolytica infection (amoebiasis), Entamoeba polecki infection, enterobiasis (pinworm infection), enterovirus infection (non-polio), Epstein-Barr virus infection, Escherichia coli infection, food-borne infection, foot-and-mouth disease, fungal dermatitis, gastroenteritis, group A streptococcal disease, group B streptococcal disease, Hansen's disease (leprosy), hantaviral pulmonary syndrome, Infestation with head lice (pediculosis), Helicobacter pylori infection, haematological disease, Hendra virus infection, hepatitis (HCV, HBV), herpes zoster (shingles), HIV infection, human ehrlichiosis, human parainfluenza virus infection, influenza, isosporiosis (Isospora infection), Lassa fever, leishmaniasis, Kala-azar (Kala-azar, Leishmania infection), leprosy, lice (clothes lice, head lice, crabs), Lyme disease, malaria, haemorrhagic fever caused by the Marburg virus, measles, meningitis, mosquito-borne diseases, Mycobacterium avium complex (MAC) infection, Naegleria infection, nosocomial infections, non-pathogenic intestinal amoeba infection, onchocerciasis (river blindness), opistorchosis (Opisthorchis infection), parvovirus infection, plague, PCP (Pneumocystis carinii pneumonia), polio, Q fever, rabies, respiratory syncytial virus (RSV) infection, rheumatic fever, Rift Valley fever, river blindness (onchocerciasis), rotavirus infection, roundworm infection, salmonellosis, Salmonella enteritidis, scabies, shigellosis, shingles, sleeping sickness, smallpox, streptococcal infection, tapeworm infection (Taenia infection), tetanus, toxic shock syndrome, tuberculosis, ulcers (peptic ulcer), valley fever, Vibrio parahaemolyticus infection, Vibrio vulnificus infection, viral haemorrhagic fever, warts, water-borne infectious diseases, West Nile virus infection (West Nile encephalitis), whooping cough, yellow fever, tuberculosis, leprosy and meningitis caused by mycobacteria
wherein U is —H, —P₁—R₁, —CO—Z₁, —P₂—Z₂ or

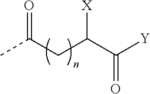

and n, X, Y, P₁, P₂, R₁, R₂, R₃, AS₁, AS₂, AS₃ and Z₁ have the meanings and preferred meanings as described herein.

Preferably, the present invention relates to the use of the compounds of general formulae (I), (Ia), (II), (IIa), (IIb), (III), (IIIa), (IIIb), (IIIc) and (IV) for the treatment and/or prevention of inflammatory diseases selected from inflammatory diseases of the central nervous system (CNS), inflammatory rheumatic diseases, inflammatory diseases of the blood vessels, inflammatory diseases of the middle ear, inflammatory bowel diseases, inflammatory diseases of the skin, inflammatory disease uveitis and inflammatory diseases of the larynx
wherein U is —H, —P₁—R₁, —CO—Z₁, —P₂—Z₂ or

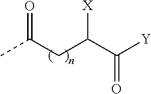

and n, X, Y, P₁, P₂, R₁, R₂, R₃, AS₁, AS₂, AS₃ and Z₁ have the meanings and preferred meanings as described herein.

Preferably, the present invention relates to the use of the compounds of general formulae (I), (Ia), (II), (IIa), (IIb), (III), (IIIa), (IIIb), (IIIc) and (IV) for the treatment and/or prevention of respiratory diseases selected from bronchial asthma, paediatric asthma, severe asthma, acute asthma attacks, chronic bronchitis, COPD (chronic obstructive pulmonary disease) and interstitial lung diseases, such as pneumonia, radiation-induced pneumonitis or fibrosis, collagenoses such as lupus erythematosus, systemic scleroderma or sarcoidosis, granulomatoses such as Boeck's disease, idiopathic interstitial pneumonia or idiopathic pulmonary fibrosis (IPF).

Preferably, the present invention relates to the use of the compounds of the general formulae (I), (Ia), (II), (IIa), (IIb), (III), (IIIa), (IIIb), (IIIc) and (IV) for the treatment and/or prevention of metabolic diseases selected from adrenogenital syndrome, Alkaptonuria, alpha-1-antitrypsin deficiency, diabetes mellitus (sugar disease), erythropoietic protoporphyria (disease from the group of porphyrias), galactosaemia, hypophosphatasia (Rathbuin syndrome), Hypothyroidism (underactive thyroid), ketoacidosis, ketosis (acetonaemia, acetonuria), Lesch-Nyhan syndrome (hyperuricaemia syndrome or hyperuricosis), methylmalonic aciduria (MMA), myoadenylate deaminase deficiency (MADD), Addison's disease (hypadrenocorticism), Conn's disease (hyperaldosteronism), Cushing's disease, Fabry's disease, Gaucher's disease, Hunter's disease (mucopolysaccharidosis type II), cystic fibrosis, phenylketonuria, porphyrias, thesaurismosis (storage disease) and uricopathy (gout)
wherein U is —H, —P₁—R₁, —CO—Z₁, —P₂—Z₂ or

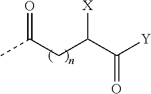

and n, X, Y, P₁, P₂, R₁, R₂, R₃, AS₁, AS₂, AS₃ and Z₁ have the meanings and preferred meanings as described herein.

Preferably, the present invention relates to the use of the compounds of the general formulae (I), (Ia), (II), (IIa), (IIb), (III), (IIIa), (IIIb), (IIIc) and (IV) for the treatment and/or prevention of the condition after surgery, after organ and tissue and bone marrow transplants, after plastic surgery operations, in particular those with systemic anaesthesia, after therapeutic irradiation with various external physical sources (such as α-, β-, γ-positrons) as well as diagnostics and therapy with radionuclide drugs administered in vivo
wherein U is —H, —P₁—R₁, —CO—Z₁, —P₂—Z₂ or

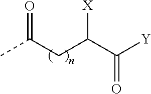

and n, X, Y, P₁, P₂, R₁, R₂, R₃, AS₁, AS₂, AS₃ and Z₁ have the meanings and preferred meanings as described herein.

In a preferred embodiment, the present invention relates to compounds of the general formulae (I), (Ia), (II), (IIa), (IIb), (III), (IIIa), (IIIb), (IIIc) and (IV) for use in the treatment of ischaemia in combination with an elevated CRP blood level. Ischaemia hinders or interrupts cellular metabolism. The ischaemia caused by the restriction or interruption of blood flow is accompanied by a lack of oxygen in the affected area. This can lead to necrosis and an infarct.

In a preferred embodiment, the compound of general formulae (I), (Ia), (II), (IIa), (IIb), (III), (IIIa), (IIIb), (IIIc) and (IV) is used in the treatment and/or prevention of diseases selected from cardiovascular arrest, stroke and pancreatitis and having temporarily acutely high CRP blood levels
wherein U is —H, —P₁—R₁, —CO—Z₁, —P₂—Z₂ or

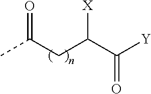

and n, X, Y, P₁, P₂, R₁, R₂, R₃, AS₁, AS₂, AS₃ and Z₁ have the meanings and preferred meanings as described herein.

An “acutely high CRP blood level” is present when the CRP concentration in the blood is well above 20 mg/L. Particularly in acute viral infections such as SARS-CoV2, CRP blood levels of around 500 mg/L or higher have been measured over a period of 3±2 days.

In another preferred embodiment, the present invention relates to compounds of general formulae (I), (Ia), (II), (IIa), (IIb), (III), (IIIa), (IIIb), (IIIc) and (IV) for use in the treatment and/or prevention of autoimmune diseases, wherein the autoimmune disease is selected from rheumatoid arthritis, inflammatory bowel disease, lupus, asthma, diabetes rheumatic diseases, AIDS, rejection of transplanted organs and tissues, rhinitis, chronic obstructive pulmonary disease, osteoporosis, ulcerative colitis, sinusitis, lupus erythematosus, recurrent infections, atopic dermatitis/eczema and occupational allergies, food allergies, drug allergies, severe anaphylactic reactions, anaphylaxis, manifestations of allergic diseases, primary immunodeficiency, antibody deficiency states, cell-mediated immunodeficiency, severe combined immunodeficiency, DiGeorge syndrome, hyper-IgE syndrome, Wiskott-Aldrich syndrome, ataxia teleangiectasia, immune-mediated forms of cancer, leukocyte defects, multiple sclerosis (MS), immune-mediated or type 1 diabetes mellitus, immune-mediated glomerulonephritis, scleroderma, pernicious anaemia, Alopecia, pemphigus, pemphigus vulgaris, myasthenia gravis, inflammatory bowel disease, Crohn's disease, psoriasis, autoimmune thyroid disease, Hashimoto's thyroiditis, dermatomyositis, Goodpasture's syndrome, myasthenia gravis pseudoparalytica, ophthalmia sympathica, phacogenic uveitis, chronic aggressive hepatitis, primary biliary cirrhosis, autoimmune haemolytic anaemia and Werlhof's disease
wherein U is —H, —P₁—R₁, —CO—Z₁, —P₂—Z₂ or

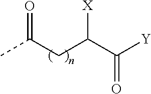

and n, X, Y, P₁, P₂, R₁, R₂, R₃, AS₁, AS₂, AS₃ and Z₁ have the meanings and preferred meanings as described herein.

In a further preferred embodiment, the present invention relates to compounds of the general formulae (I), (Ia), (II), (IIa), (IIb), (III), (IIIa), (IIIb), (IIIc) and (IV) for use in the treatment and/or prevention of infectious diseases, wherein the infectious diseases are caused, induced, initiated and/or aggravated by bacteria, viruses, prions, parasites, fungi and/or triggered by irritative, traumatic, metabolic, allergic, autoimmunological or idiopathic causes
wherein U is —H, —P₁—R₁, —CO—Z₁, —P₂—Z₂ or Y

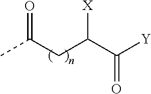

and n, X, Y, P₁, P₂, R₁, R₂, R₃, AS₁, AS₂, AS₃ and Z₁ have the meanings and preferred meanings as described herein.

In a further preferred embodiment, the present invention relates to compounds of the general formulae (I), (Ia), (II), (IIa), (IIb), (III), (IIIa), (IIIb), (IIIc) and (IV) for use in the treatment and/or prevention of infectious diseases, wherein the infectious disease is a disease caused by coronaviruses, in particular SARS-CoV-2, and a temporarily acutely high or chronically elevated CRP blood level is diagnosed
wherein U is —H, —P₁—R₁, —CO—Z₁, —P₂—Z₂ or

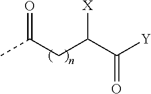

and n, X, Y, P₁, P₂, R₁, R₂, R₃, AS₁, AS₂, AS₃ and Z₁ have the meanings and preferred meanings as described herein.

In a further embodiment, the compounds of the general formulae (I), (Ia), (II), (IIa), (IIb), (III), (IIIa), (IIIb), (IIIc) and (IV) described herein can be used in combination with extracorporeal methods for lowering the CRP level, such as dialysis or apheresis. In these procedures, CRP is removed from blood plasma by affinity chromatography using columns loaded with CRP-binding material. The combination is useful for treating particularly high CRP levels above 550 mg/L. Thus, the present invention relates to compounds of the general formulae (I), (Ia), (II), (IIa), (IIb), (III), (IIIa), (IIIb), (IIIc) and (IV) for use as a medicament for binding and/or neutralising C-reactive protein in the blood in combination with an apheresis or dialysis procedure for lowering the C-reactive protein level
wherein U is —H, —P₁—R₁, —CO—Z₁, —P₂—Z₂ or

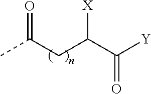

and n, X, Y, P₁, P₂, R₁, R₂, R₃, AS₁, AS₂, AS₃ and Z₁ have the meanings and preferred meanings as described herein.

A further aspect of the present invention relates to pharmaceutical compositions comprising at least one compound of the general formulae (I), (Ia), (II), (IIa), (IIb), (III), (IIIa), (IIIb), (IIIc) und (IV)
wherein U is —H, —P₁—R₁, —CO—Z₁, —P₂—Z₂ or

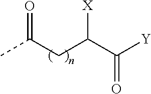

and n, X, Y, P₁, P₂, R₁, R₂, R₃, AS₁, AS₂, AS₃ and Z₁ have the meanings and preferred meanings described herein.

For use as a medicament for binding and/or neutralising C-reactive protein in blood, the pharmaceutical formulation may further comprise pharmaceutically acceptable carriers, excipients and/or diluents. The pharmaceutical compositions described herein may be prepared in a conventional solid or liquid carrier or diluent and a conventional pharmaceutically prepared excipient at a suitable dosage in a known manner.

The pharmaceutical compositions described herein are typically administered together with suitable acceptable carriers selected with respect to the intended form of administration, e.g., as powders for constitution, gels, elixirs, dispersible granules, syrups, suspensions, and the like, and in accordance with conventional pharmaceutical practices.

Also included are preparations in solid form that are intended to be converted into a liquid form shortly before use. These liquid forms include solutions, suspensions and emulsions.

The pharmaceutical compositions described herein may be administered subcutaneously, by spray, by injection, intramuscularly, as a suppository or trans(epi)dermally.

The compounds of general formulae (I), (Ia), (II), (IIa), (IIb), (III), (IIIa), (IIIb), (IIIc) and (IV) described herein and the pharmaceutical compositions thereof described herein are preferably intended for the treatment of humans, but may also be used in animals and in particular in horses and preferably riding, racing and dressage horses.

In a preferred embodiment, the compound of general formulae (I), (Ia), (II), (IIa), (IIb), (III), (IIIa), (IIIb), (IIIc) and (IV) contained in the pharmaceutical composition described herein is administered in a range of 1 mg/kg to 100 mg/kg, preferably 2 mg/kg to 100 mg/kg, preferably 5 mg/kg to 100 mg/kg, preferably 10 mg/kg to 100 mg/kg per body weight per day.

In a further preferred embodiment, the compounds of general formulae (I), (Ia), (II), (IIa), (IIb), (III), (IIIa), (IIIb), (IIIc) and (IV) as well as a pharmaceutical composition thereof may be used in combination with at least one complement blocker for binding and/or neutralising C-reactive protein in the blood. Preferably, the complement blocker inhibits the complement protein(s) C3 and/or C5 by binding to an active site of the complement protein(s) C3 and/or C5. The complement blocker also prevents complement activation, which stops the spread of damage and scarring in the ischaemic tissue and also reduces or completely prevents infarct scarring after a heart attack.

Thus, the present invention also relates to a compound of the general formulae (I), (Ia), (II), (IIa), (IIb), (III), (IIIa), (IIIb), (IIIc) and (IV) or a pharmaceutical formulation thereof for use as a medicament for binding and/or neutralising C-reactive protein in the blood, in combination with at least one complement blocker
wherein U is —H, —P₁—R₁, —CO—Z₁, —P₂—Z₂ or

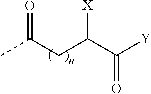

and n, X, Y, P₁, P₂, R₁, R₂, R₃, AS₁, AS₂, AS₃ and Z₁ have the meanings and preferred meanings described herein.

For the U.S.A., a method of treating a patient is now described.

This method comprises administering to a patient a compound of the general formula (I), (Ia), (II), (IIa), (IIb), (III), (IIIa), (IIIb), (IIIc) or (IV) for the treatment and/or prevention of diseases associated with and/or caused by an elevated CRP level and thus for the treatment and/or prevention of diseases selected from tumour diseases, inflammatory diseases, metabolic diseases, autoimmune diseases, respiratory diseases, cardiovascular diseases, post-operative conditions and infectious diseases.

### DESCRIPTION OF THE FIGURES

FIG. 1 ELISA for the binding of huCRP to solid phase-bound biotin-D-Glu(APPC)—NH₂ (2) with increasing CRP concentration. The amount of bound huCRP was detected using a mAb against human CRP.

FIG. 2 ELISA for the binding of free CRP to free biotin-D-Glu(APPC)—NH₂ (2). In solution, (2) was bound to free huCRP before. Fixation of (2) on streptavidin-coated MTP is used to detect the huCRP bound to (2).

FIG. 3 ELISA for the binding of huCRP to solid phase-bound biotin-PEG-L-Glu(APPC)—NH₂ (3) with increasing CRP concentration. The amount of bound huCRP was detected using a mAb against human CRP.

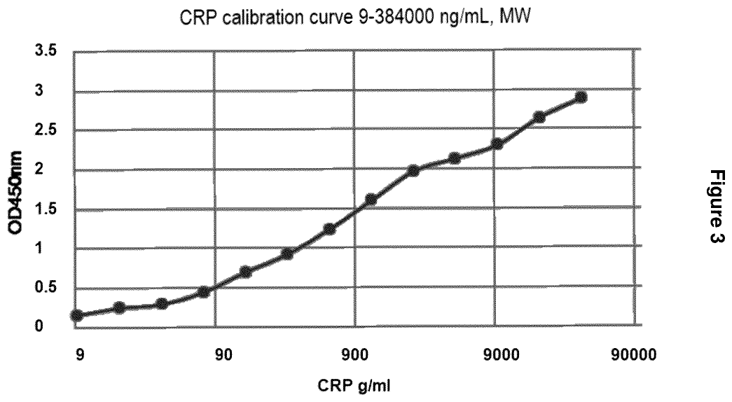

FIG. 4 Concentration-dependent inhibition of CRP binding. All tested compounds (free APPC and APPC coupled to stabilisers) showed a good inhibition capacity with regard to CRP.

### EXAMPLES

### Abbreviations

APPC: 4-aminophenylphosphocholine, ligand for CRP
eq.: equivalent
BSA: bovine serum albumin
CRP: C reactive protein
ELISA: Enzyme-Linked Immuno Sorbent Assay
HSA: human serum albumin
huCRP: human CRP
IgG: immunoglobulin G
cav.: cavity
mAb. monoclonal antibody
MTP: microtiter plate
Opp: 2-phenylisopropyl
PBS: Phosphate buffered physiological saline solution
POD-anti Maus: peroxidase, coupled to a secondary antibody against murine IgG
S: laboratory shaker for MTP
TMB: 3,3′,5,5′ Tetramethylbenzidine, substrate for POD, colour reagent
Tween20: detergent, commercial name

### Example 1: Synthesis of APPC-Bound D-Glutamic Acid Amide (D-Glu(APPC)—NH₂)=Compound 1

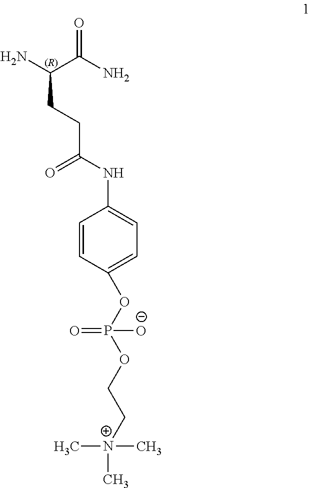

D-Glu(APPC)—NH₂ (1) was synthesised as follows:

D-glutamic acid amide was activated in solution in the presence of 1 eq. 1-ethyl-3-(3-dimethylaminopropyl)carbodiimide and then 1 eq. 4-aminophenylphosphorylcholine was added. The reaction mixture was stirred overnight and then worked up. Compound 1 was obtained almost quantitatively as a colourless solid.

### Example 2: Synthesis of Biotinylated APPC-Bound D-Glutamic Acid Amide (Biotin-D-Glu(APPC)—NH₂)=Compound 2

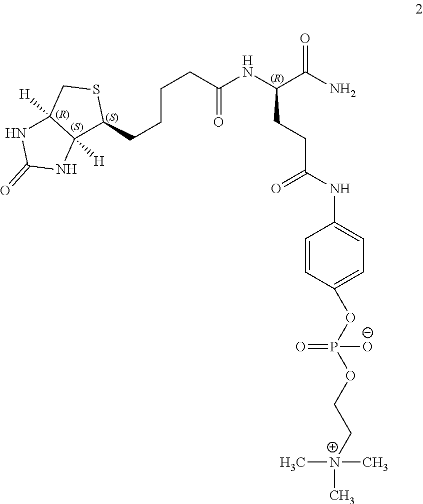

Biotin-D-Glu(APPC)—NH₂ (2) was synthesised analogously to (1) as follows: Biotin-D-glutamic acid amide was activated in solution in the presence of 1 eq. 1-ethyl-3-(3-dimethylaminopropyl)carbodiimide and then 1 eq. 4-aminophenyl-phosphorylcholine was added. The reaction mixture was stirred overnight and then worked up. Compound 2 was obtained almost quantitatively as a colourless solid.

### Example 3: Assay for the Binding of CRP to Solid Phase-Fixed Biotin-D-Glu(APPC)—NH₂ (Compound 2)

Streptavidin-coated microtiter plates were incubated with a solution containing biotin-D-Glu(APPC)—NH₂) (2) and then washed with phosphate buffer (PBS). The reactive parts of the MTP surface were then blocked with BSA solution (a standard procedure). CRP was then added in a concentration range of 31-500 ng/ml and incubated (see table below). After renewed washing, the bound CRP was detected using a murine monoclonal antibody (DH7) against CRP and a secondary, POD-conjugated anti-mouse antibody, followed by substrate development

TABLE 1
Data of the underlying CRP binding curve (mean values from duplicate
determinations)

| | Optical |
|---|---|
| CRP [ng/ml] | Density (OD) |
| 500 | 3.242 |
| 250 | 2.627 |
| 125 | 2.081 |
| 62.5 | 1.512 |
| 31 | 1.061 |
| 0 | 0.173 |

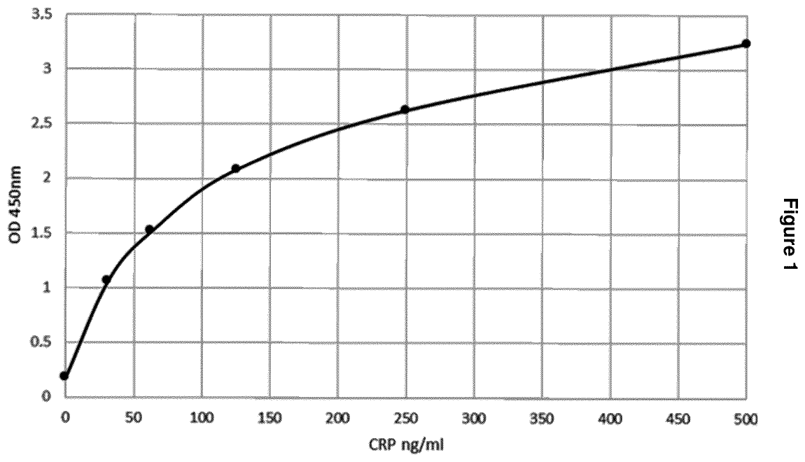

The CRP binding curve in FIG. 1 shows that compound 2 binds to CRP. The compound therefore fulfils the requirements of a low-molecular pharmacological CRP blocker.

### Example 4: Assay for the Binding of Free CRP and Free Biotin-D-Glu(APPC)—NH₂ (2)

CRP and compound 2 were incubated in a reaction vessel for 1 h at room temperature for complexation. The final concentrations were 10 μg/ml CRP (MW approx. 125 KD) and 2 μg/ml compound 2 (MW approx. 1 KD). The solution was then added to streptavidin-coated microtiter plates (MTP) at the dilution levels indicated in Tab. 2 at 100 μl/ml each and incubated with a block solution (Roche) for 30 min at room temperature. The MTPs were then washed 3 times. Using the in house monoclonal antibody “DH7” specific for huCRP and a peroxidase-conjugated secondary antibody (anti-mouse) as well as the substrate TMB, the huCRP previously bound to compound 2 was detected.

TABLE 2
Dilution series CRP: 2 and its optical density (mean values from duplicate
determinations)

| complex dilution | Optical Density |
|---|---|
| CRP:compound 2 | (OD) |
| 1:20 | 2.685 |
| 1:40 | 2.219 |
| 1:80 | 1.801 |
| 1:160 | 1.242 |
| 1:320 | 0.875 |
| 1:640 | 0.548 |
| 1:1280 | 0.381 |
| 0 | 0.144 |

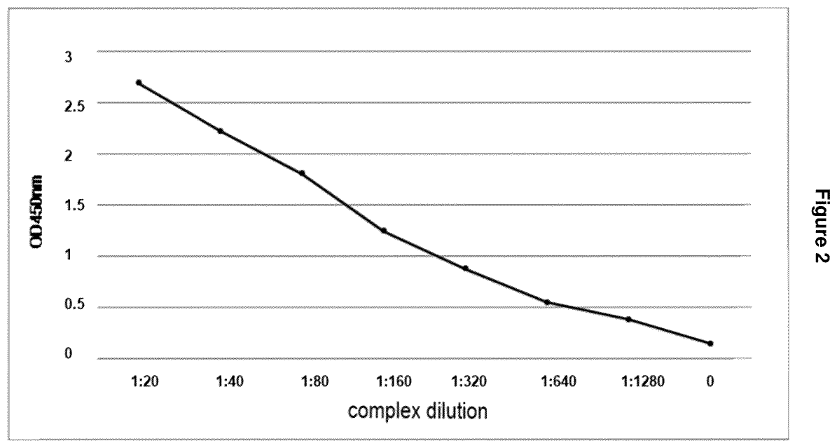

Compound 2 binds CRP in solution and as a complex to the streptavidin of the microtiter plate. This is also evident from the almost linear dilution series in FIG. 2.

### Example 5: Synthesis of PEGylated L-glutamic acid (biotin-PEG-L-Glu(APPC)—NH₂) Compound 3′

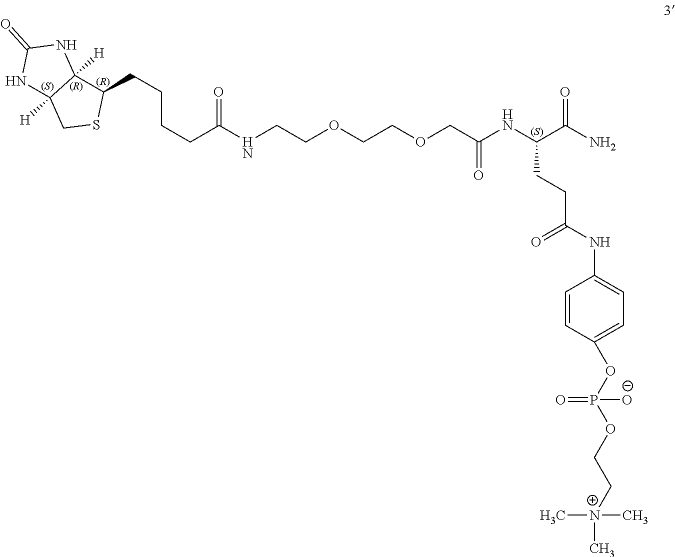

Compound 3′ was synthesised under standard Fmoc solid phase synthesis conditions as follows:
Fmoc-L-Glu(Opp)-OH was bound to solid phase (TentaGel® HL RAM, 0.37 mMol/g). The Fmoc group was cleaved by addition of piperidine and then biotin-PEG₂-NHS
(9-(biotinamido)-4,7-dioxanonanoic acid-N-succinimide ester) was added. The 2-phenylisopropyl group (Opp) was cleaved off by addition of acid, followed by addition of 4-aminophenylphosphorylcholine and HATU (O-(7-azabenzotriazol-1-yl)-N,N,N′,N′-tetramethyluronium hexafluorophosphate]) in the presence of a base.

The resin was then separated using TFA, water and triethylsilane. After purification by preparative HPLC (C18), compound 3′ was obtained with a purity >95%.

### Example 6: Assay for the Binding of Free CRP to Solid Phase-Fixed (Biotin-PEG-L-Glu(APPC)—NH₂) (Compound 3′)

The assay was performed in 7 steps. A schematic sequence is shown in Table 4:

TABLE 3
Optical density (OD at 450 nm) of the binding of increasing CRP
concentrations to fixed 3; MW = mean value.

| CRP | | | |
|---|---|---|---|
| ng/ml | OD | OD | MW |
| 38400 | 2.824 | 2.944 | 2.884 |
| 19200 | 2.653 | 2.626 | 2.6395 |
| 9600 | 2.273 | 2.316 | 2.2945 |
| 4800 | 2.057 | 2.193 | 2.125 |
| 2400 | 1.936 | 1.989 | 1.9625 |
| 1200 | 1.563 | 1.632 | 1.5975 |
| 600 | 1.221 | 1.249 | 1.235 |
| 300 | 0.881 | 0.96 | 0.9205 |
| 150 | 0.669 | 0.716 | 0.6925 |
| 75 | 0.428 | 0.458 | 0.443 |
| 37.5 | 0.28 | 0.299 | 0.2895 |
| 18.7 | 0.214 | 0.27 | 0.242 |
| 9.3 | 0.147 | 0.16 | 0.1535 |

TABLE 4
Schematic sequence of the experiment

| | | | μl/ | | | |
|---|---|---|---|---|---|---|
| No | reagent | conc.. | cav. | buffer | incubation | washing |
| 1 | Compd. 3 (2 μg/ml) into | final 2 μg/ml, | 100 | 0.05M PBS + 0.01% | 1 h rt + S | 2x PBS/Tween |
| | each well of streptavidin- | dissolved with | | BSA + 0.045% | | |
| | coated microtiter plates | 8 μl DMSO, | | ProClin 300*, pH 7.2) | | |
| | (standard capacity) from | then + 1 ml | | 0.05M PBS = 250 ml | | |
| | BIOTEZ Berlin | PBS | | water + 0.47 g | | |
| | | aliquoted and | | KH₂PO₄ + 1.62 g | | |
| | | lyophilised | | Na₂HPO₄*2H₂O + | | |
| | | again | | 2.2 g NaCl | | |
| 2 | CRP (1.27 mg/ml) | 38.400 − 0 | 100 | 25 mM Tris/HCl | 1 h rt + S | 3x |
| | | ng/ml | | (pH 8.0), | | |
| | | | | 0.9% NaCl, 0.1% | | |
| | | | | Tween 20, 0.1% BSA, | | |
| | | | | 5 mM CaCl₂ | | |
| 3 | monoclonal antibody | final | 100 | 25 mM Tris/HCl | 1 h rt + S | 3x |
| | (DH7) against CRP | 5 μg/ml | | (pH 7.3), | | |
| | (purified 500 μg/ml) | | | 0.9% NaCl, 0.1% | | |
| | (dilution 1:100) | | | Tween 20, 0.1% BSA | | |
| 4 | POD-anti mouse IgG | 1:3000 | 100 μl | POD anti mouse IgG | 1 h rt + S | 3x |
| | (Sigma) | | | with CRP buffer pH | | |
| | | | | 7.3 | | |
| 5 | substrate: TMB Seramun | Ready for use | 100 | 100 μl | 13 ± 2 min, rt, | |
| | | | | SeramunBlaufast | in dark | |
| 6 | stop solution: H₂SO₄ | 0.25M | 50 | 0.25M H₂SO₄ | | |

| | |
|---|---|
| 7 | Measurement of the optical density (OD) of the colour solution: 450 nm (Ref.: 620 nm) |

*ProClin 300: 1:1 mixture of 5-chloro-2-methyl-1,2-thiazol-3-one and 2-methyl-1,2-thiazol-3-one

1. In streptavidin-coated microtiter plates, 100 μl of a solution of 3 (final concentration 2 μg/ml) was added to each well and incubated for 1 hour (h) at room temperature (rt, 20-22° C.). The wells were then washed twice with 200 μl PBS/Tween buffer each.
2. Freshly prepared human CRP was added to the wells (100 μl/ml each) from a stock solution (1.27 mg/ml in physiological saline) in dilution steps halving the primary concentration (dilution buffer: 25 mM Tris/HCl, pH 8.0) in the range of 38,400-9.3 ng/ml. The microtiter plates were incubated for 1 h at rt and washed 3 times with 200 μl PBS/Tween buffer to remove the excess CRP.
3. The bound CRP was detected using a purified murine monoclonal antibody directed against human CRP (mAb). The mAb was added in a final concentration of 5 μg/ml (dilution buffer: 25 mM Tris/HCl) to 100 μl each and incubated for 1 h at rt. The wells of the MTP were then washed 3 times to remove the excess, unbound mAb.
4. The CRP bound to 3 was labelled by incubating the wells with 100 μl peroxidase-labelled antibody against mouse IgG (1:3,000 dilution, Sigma) in “CRP buffer”. The incubation time was 1 h at rt. The excess antibody was removed by washing the wells 3 times with “CRP buffer”.
5. The substrate for the peroxidase (POD) was 100 μl 3,3′,5,5′-tetramethylbenzidine (TMB, Seramun, SeramunBlau®fast) per well. The colourless substrate molecule was converted by POD into a photometrically measurable blue colour complex. The substrate incubation was carried out in the dark within 12±2 min at rt.
6. The enzymatic reaction was interrupted by the addition of 50 μl of 0.25 M sulphuric acid.
7. Immediately afterwards, the optical density was measured photometrically in a microtiter plate photometer at a wavelength of 450 nm (against a reference of 620 nm). The colour intensity as optical density (OD) corresponds to the amount of CRP present. Results are shown in Table 3.

### Example 7: Synthesis of Double PEGylated D-Glutamic Acid (PEG-L-Glu(APPC)-PEG-NH₂)=Compound 4

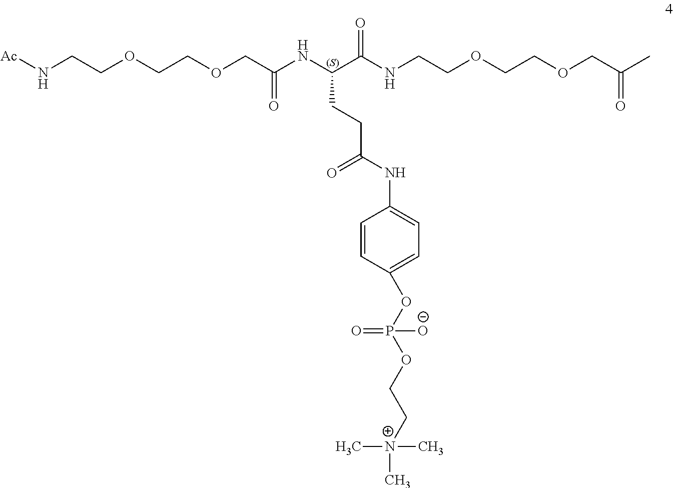

Compound 4 was prepared analogue to compound 3 according to the standard conditions of the Fmoc solid phase synthesis.

### Example 8: Assay for the Binding of Free CRP to Solid Phase-Fixed

A microtiter plate was coated with 100 μg/mL of a BSA-APPC construct, which binds CRP in a calcium-dependent manner, for 1 hour at room temperature (coating buffer: 0.1 M NaHCO₃). Subsequently, 4 washes with 400 μl binding buffer (0.1 M Tris, 0.2 M NaCl, 2 mM CaCl₂)) and non-specific binding sites were saturated with block solution (1% casein, 0.9% NaCl, 0.001% thiomersal) for 1 h at room temperature or overnight at 2-8° C.

Afterwards, the block solution was removed and the samples were applied in binding buffer. CRP-containing human plasma was diluted to 50 ng/mL CRP and applied either pure or together with different concentrations (0.25-3 mM) of the various inhibitors 1, 4, 5 and APPC and incubated for 1.5 h at room temperature with horizontal shaking (600 rpm). The plate was then washed again as described above.

After washing, 100 μL of a POD-labelled rabbit anti-huCRP antibody (final concentration: 25 ng/mL) in calcium-containing block solution (final concentration of calcium 4 mM) was added and the plate was incubated for 1.5 h at room temperature with shaking.

The plate was then washed again as described above. Development was carried out by adding 100 μl substrate buffer (0.2 M citric acid, 0.01% H₂O₂, 100 μg/mL TMB). The colour reaction was stopped by adding 50 μL of 1 M H₂SO₄ and the absorbance was measured at 450 nm. The absorbance at 655 nm (turbidity) was subtracted as background.

### Result

For the determination of inhibition, the OD of the CRP with inhibitor was normalised to the OD of the CRP without inhibitor:

$${OD}_{relative} = \frac{{OD}_{with inhibitor}}{{OD}_{without inhibitor}}$$

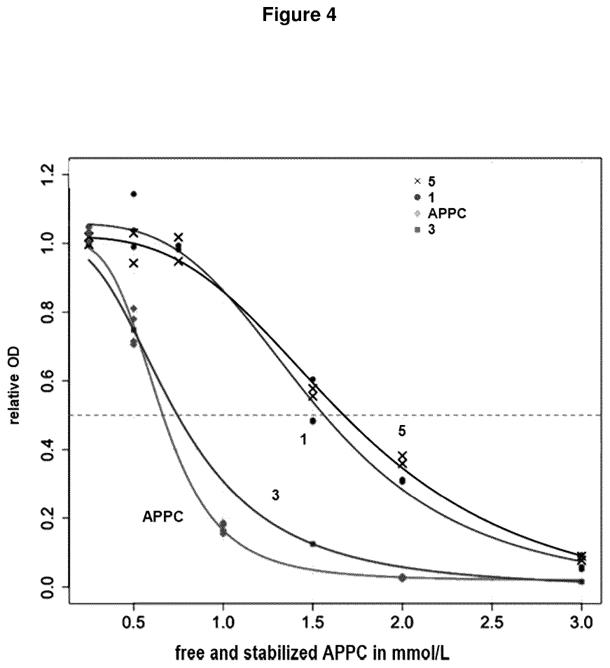

The relative OD was then plotted against the concentration of stabilised APPC constructs in the batch. This results in the curve shown in FIG. 4. Only APPC was used as a comparative sample. It is clear that constructs 1 and 5 or 3 and APPC show an inhibition curve that is identical within the measurement uncertainty. Mathematically, this results in a 50% inhibitor concentration (IC₅₀) of 1.6±0.1 mmol/L for 1/5. For APPC/3, the corresponding concentration is 0.7±0.06 mmol/L. Thus, all stabilised APPC constructs show a good inhibition capacity.

What is claimed is:

1. A compound of general formula (I): 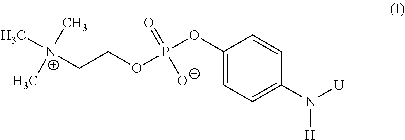
wherein U is selected from —P₁—R₁, —CO—Z₁, —P₂—Z₂ or 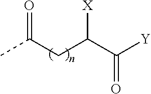
wherein n is an integer selected from 1, 2 and 3;
X represents —NH₂, —NH—P₁—R₁, —NH—CO—Z₁ or —NH—P₁—Z₁;
Y represents —OH, —NH₂, —P₂—R₂, —NH—Z₂ or —P₂—Z₂;
R₁ is selected from —H, —CH₃, —CH₂CH₃, —CO—CH₃ and —CO—CH₂CH₃;
R₂ is selected from —OH, —OCH₃, —OCH₂CH₃, —CH₃, and —NH₂;
P₁ is selected from -Q₁-, -Q₁-Q₂-, -Q₁-Q₂-Q₃- and -Q₁-Q₂-Q₃-Q₄-;
P₂ is selected from -Q₅-, -Q₅-Q₆-, -Q₅-Q₆-Q₇- and -Q₅-Q₆-Q₇-Q₈-; 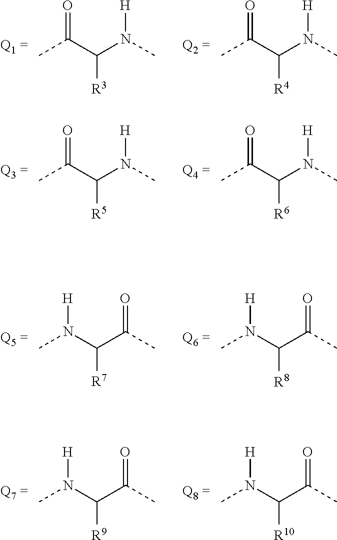
wherein R₃, R₄, R₅, R₆, R₇, R₈, R₉ and R₁₀ represent independently of each other —H, —CH₃, —CH₂OH, —CH₂SH, —COOH, —CONH₂, —CH(OH)CH₃,
—CH(CH₃)₂, —CH₂—CH(CH₃)₂, —CH(CH₃)—C₂H₅, —CH₂—C₆H₅, —CH₂—C₆H₄—OH, —C₂H₄—S—CH₃, —CH₂—COOH, —CH₂—CONH₂, —C₂H₄—COOH, —C₂H₄—CONH₂, —C₃H₆—NH—C(NH)—NH₂, —C₄H₈—NH₂, 5-imidazolyl or 2-indolyl, or R₃, R₄, R₅, R₆, R₇, R₈, R₉ and R₁₀ and the neighbouring nitrogen atom together with the atoms to which they are bound form a pyrrolidine ring;
Z₁ means 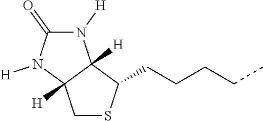
or
—(CH₂)ₒ₁—(O—C₂H₄)ₒ₂—O—(CH₂)ₒ₃-T¹, wherein
o1 is an integer selected from 1, 2, 3, 4, 5 and 6;
o2 is an integer selected from 0, 1, 2, 3, 4, 5, 6, 7, 8, 9 and 10;
o3 is an integer selected from 1, 2, 3, 4, 5 and 6;
Z₂ means —(CH₂)ₒ₄—(O—C₂H₄)ₒ₅—O—(CH₂)ₒ₆-T², wherein
o4 is an integer selected from 1, 2, 3, 4, 5 and 6;
o5 is an integer selected from 0, 1, 2, 3, 4, 5, 6, 7, 8, 9 and 10;
o6 is an integer selected from 1, 2, 3, 4, 5 and 6;
T¹ means —H, —NH₂, —OCH₃, —CO—CH₃, —CO—OCH₃, —CO—NH₂, —NH—CO—CH₃ or 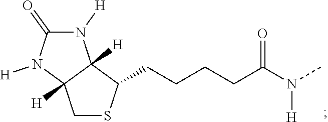
T² means —H, —NH₂, —OCH₃, —CO—CH₃, —CO—OCH₃, —CO—NH₂ or
—NH—CO—CHs;
as well as enantiomers, stereoisomeric forms, mixtures of enantiomers, diastereomers, mixtures of diastereomers, hydrates, solvates, tautomers, racemates of the aforementioned compounds and pharmaceutically acceptable salts thereof.

2. The compound according to claim 1 having the structure of general formula (II) 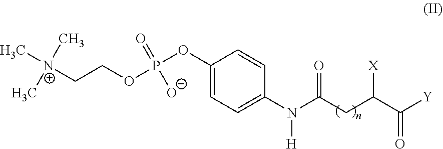
wherein n is 2, and X and Y have the meanings defined in claim 1.

3. The compound according to claim 2 having the structure of general formula (II),
wherein n is 2,
Y represents —OH or —NH₂,
X represents —NH₂ or —NH—CO—Z₁, wherein
Z₁ is 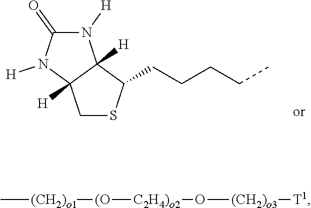
o1 is 1 or 2,
o2 is an integer selected from 1, 2, 3, 4 and 5;
o3 is 1 or 2 and
T¹ is —NH—CO—CH₃,

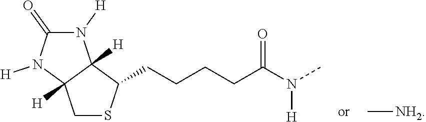

4. The compound according to claim 2 having the structure of general formula (II),
wherein n is 2,
X means —NH₂ or —NH—P₁—R₁,
Y means —OH, —NH₂ or —P₂—R₂,
P₁ and P₂ mean independently of each other -AS₁-, -AS₁-AS₂- or -AS₁-AS₂-AS₃- and
AS₁, AS₂ and AS₃ represent for each occurrence independently of each other amino acids of the following structure: 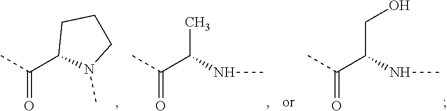
R₁ and R₂ have the meanings as defined in claim 1.

5. The compound according to claim 2 having the structure of general formula (II),
wherein n is 2, Y is —OH, and X is —NH₂.

6. The compound according to claim 2 having the structure of general formula (II),
wherein n is 2,
X represents —NH—P₁—R₁,
Y represents —P₂—R₂,
P₁ is selected from -Q₁-Q₂-, -Q₁-Q₂-Q₃- and -Q₁-Q₂-Q₃-Q₄-;
P₂ is selected from -Q₅-Q₆-, -Q₅-Q₆-Q₇- and -Q₅-Q₆-Q₇-Q₈-;
and R₁, R₂ as well as Q₁-Q₅ have the meanings as defined in claim 1.

7. The compound according to claim 1 having the structure of general formula (III) 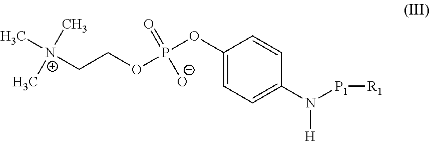
wherein P₁ represents -Q₁-, -Q₁-Q₂-, -Q₁-Q₂-Q₃- and -Q₁-Q₂-Q₃-Q₄-, with 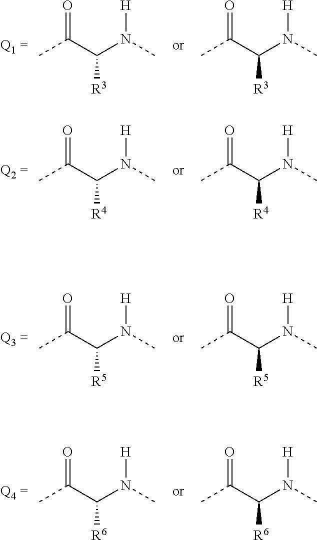
and R₁, R₃, R₄, R₅ and R₆ have the meanings as defined in claim 1.

8. A pharmaceutical composition containing at least one compound according to claim 1.

9. The pharmaceutical composition according to claim 8 further comprising a pharmaceutically acceptable carrier, an excipient and/or a diluent.

10. A method for the treatment or prevention of a disease caused by or associated with a CRP blood level of >20 mg/L, comprising administering to a patient a compound of general formula (I) or a pharmaceutical
composition containing at least one compound of general formula (I) 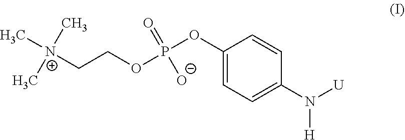
wherein U is —H, —P₁—R₁, —CO—Z₁, —P₂—Z₂ or 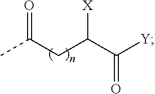
n is an integer selected from 1, 2 and 3;
X means —NH₂, —NH—P₁—R₁, —NH—CO—Z₁ or —NH—P₁—Z₁;
Y means —OH, —NH₂, —P₂—R₂, —NH—Z₂ or —P₂—Z₂;
R₁ is selected from —H, —CH₃, —CH₂CH₃, —CO—CH₃ and —CO—CH₂CH₃;
R₂ is selected from —OH, —OCH₃, —OCH₂CH₃, —CH₃, and —NH₂;
P₁ is selected from -Q₁-, -Q₁-Q₂-, -Q₁-Q₂-Q₃- and -Q₁-Q₂-Q₃-Q₄-;
P₂ is selected from -Q₅-, -Q₅-Q₆-, -Q₅-Q₆-Q₇- and -Q₅-Q₆-Q₇-Q₈-; 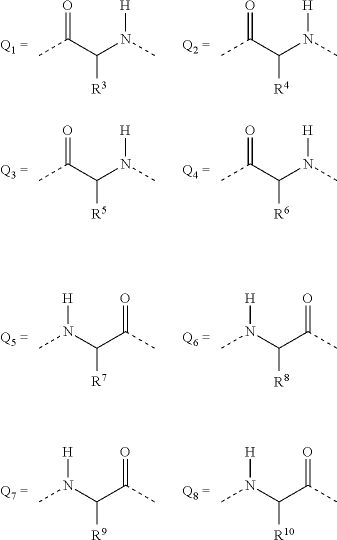
wherein R³, R⁴, R⁵, R⁶, R⁷, R⁸, R⁹ and R¹⁰ represent independently of each other —H, —CH₃, —CH₂OH, —CH₂SH, —COOH, —CONH₂, —CH(OH)CH₃, —CH(CH₃)₂, —CH₂—CH(CH₃)₂, —CH(CH₃)—C₂H₅, —CH₂—C₆H₅,
—CH₂—C₆H₄—OH, —C₂H₄—S—CH₃, —CH₂—COOH, —CH₂—CONH₂, —C₂H₄—COOH, —C₂H₄—CONH₂, —C₃H₆—NH—C(NH)—NH₂, —C₄H₈—NH₂, 5-imidazolyl or 2-indolyl, or R³, R⁴, R⁵, R⁶, R⁷, R⁸, R⁹ and R¹⁰ and the neighbouring nitrogen atom together with the atoms to which they are bound form a pyrrolidine ring;
Z₁ means 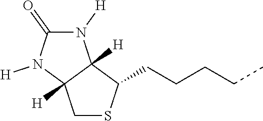
or
—(CH₂)ₒ₁—(O—C₂H₄)ₒ₂—O—(CH₂)ₒ₃-T¹, wherein
o1 is an integer selected from 1, 2, 3, 4, 5 and 6;
o2 is an integer selected from 0, 1, 2, 3, 4, 5, 6, 7, 8, 9 and 10;
o3 is an integer selected from 1, 2, 3, 4, 5 and 6;
Z₂ means —(CH₂)ₒ₄—(O—C₂H₄)ₒ₅—O—(CH₂)ₒ₆-T², wherein
o4 is an integer selected from 1, 2, 3, 4, 5 and 6;
o5 is an integer selected from 0, 1, 2, 3, 4, 5, 6, 7, 8, 9 and 10;
o6 is an integer selected from 1, 2, 3, 4, 5 and 6;
T¹ means —H, —NH₂, —OCH₃, —CO—CH₃, —CO—OCH₃, —CO—NH₂, —NH—CO—CH₃ or 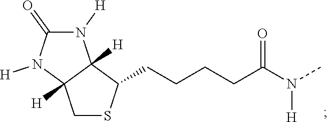
T² means —H, —NH₂, —OCH₃, —CO—CH₃, —CO—OCH₃, —CO—NH₂ or
—NH—CO—CHs;
as well as enantiomers, stereoisomeric forms, mixtures of enantiomers, diastereomers, mixtures of diastereomers, hydrates, solvates, tautomers, racemates of the aforementioned compounds and pharmaceutically acceptable salts thereof, sufficient to treat or prevent the disease.

11. The method according to claim 10, wherein the disease is selected from tumour diseases, inflammatory diseases, autoimmune diseases, respiratory diseases, metabolic diseases, vascular occlusions, cardiovascular diseases, post-operative conditions and infectious diseases.

12. The method according to claim 10, wherein the disease is a heart attack, cardiovascular arrest, stroke or pancreatitis.

13. The method according to claim 12 in combination with an apheresis procedure or dialysis procedure to lower the level of C-reactive protein.

14. The method according to claim 10 in combination with at least one complement blocker.

15. The method according to claim 10 in combination with at least one CRP-binding phosphocholine conjugate comprising a 4-aminophenylphosphorylcholine covalently bound to human serum albumin.
